(19) United States
(12) Patent Application Publication
(10) Pub. No.: US 2004/0096465 A1
(43) Pub. Date: May 20, 2004

(54) Novel receptors for $1(helicobater pyroli) and use thereof

(76) Inventors: Halina Miller-Podraza, (US); Susann Teneberg, Hind?aring;s (SE); Jonas Angström, Goteb?ouml;rg (SE); Karl-Anders Karlsson, G?ouml;teborg (SE); Jari Natunen, Vantaa (SE)

Correspondence Address: BIRCH STEWART KOLASCH & BIRCH, PO BOX 747, FALLS CHURCH, VA, 22040-0747, US

(21) Appl. No.: 10/466,415

(22) PCT Filed: Jan. 18, 2002
(86) PCT No.: PCT/FI02/00043

(30) Foreign Application Priority Data
Jan. 19, 2001 (FI) 20010118

(51) Int. Cl. A61K 39/02; A61K 31/739

(52) U.S. Cl. 424/234.1; 514/54; 536/53

(57) ABSTRACT

The present invention describes a substance or a receptor comprising Helicobacter pylori binding oligosaccharide sequence [Gal(A)_{q}(NAc)ᵣ/Glc(A)_{q}(NAc)ᵣα3/β3]ₛ[Galβ4GlcNAcβ3]ₜGalβ4Glc(NAc)ᵤ wherein q, r, s, t, and u are each independently 0 or 1, and the use thereof in, e.g., pharmaceutical and nutritional compositions for the treatment of conditions due to the presence of Helicobacter pylori. The invention is also directed to the use of the receptor for diagnostics of Helicobacter pylori.

### FIELD OF THE INVENTION

The present invention describes a substance or receptor binding to Helicobacter pylori, and use thereof in, e.g., pharmaceutical and nutritional compositions for the treatment of conditions due to the presence of Helicobacter pylori. The invention is also directed to the use of the receptor for diagnostics of Helicobacter pylori.

### BACKGROUND OF THE INVENTION

Helicobacter pylori has been implicated in several diseases of the gastrointestinal tract including chronic gastritis, non-steroidal anti-inflammatory drug (NSAID) associated gastric disease, duodenal and gastric ulcers, gastric MALT lymphoma, and gastric adenocarcinoma (Axon, 1993; Blaser, 1992; DeCross and Marshall, 1993; Dooley, 1993; Dunn et al., 1997; Lin et al., 1993; Nomura and Stemmermann, 1993; Parsonnet et al. 1994; Sung et al., 2000 Wotherspoon et al., 1993). Totally or partially non-gastrointestinal diseases include sudden infant death syndrome (Kerr et al., 2000 and U.S. Pat. No. 6,083,756), autommune diseases such as autoimmune gastritis and pernicious anaemia (Appelmelk et al., 1998; Chmiela et al, 1998; Clayes et al., 1998; Jassel et al., 1999; Steininger et al., 1998) and some skin diseases (Rebora et al., 1995), pancreatic disease (Correa et al., 1990), liver diseases including adenocarcinoma (Nilsson et al., 2000; Avenaud et al., 2000) and heart diseases such as atherosclerosis (Farsak et al., 2000). Multiple diseases caused or associated with Helicobacter pylori has been reviewed (Pakodi et al., 2000). Of prime interest with respect to bacterial colonization and infection is the mechanism(s) by which this bacterium adheres to the epithelial cell surfaces of the gastric mucosa.

Glycoconjugates, both lipid- and protein-based, have been reported to serve as receptors for the binding of this microorganism as, e.g., sialylated glycoconjugates (Evans et al., 1988), sulfatide and GM3 (Saitoh et al., 1991), Le^{b} determinants (Borén et al., 1993), polyglycosylceramides (Miller-Podraza et al., 1996; 1997a), lactosylceramide (Ångström et al., 1998) and gangliotetraosylceramide (Lingwood et al., 1992; Ångström et al., 1998). Other potential receptors for Helicobacter pylori include the polysaccharide heparan sulphate (Ascensio et al., 1993) as well as the phospholipid phosphatidylethanolamine (Lingwood et al., 1992).

US patents of Zopfet al.: U.S. Pat. No. 5,883,079 (March 1999), U.S. Pat. No. 5,753,630 (May 1998) and U.S. Pat. No. 5,514,660 (May, 1996) describe Neu5Acα3Gal- containing compounds as inhibitors of the H. pylori adhesion. The sialyl-lactose molecule inhibits Helicobacter pylori binding to human gastrointestinal cell lines (Simon et al., 1999) and is also effective in a rhesus monkey animal model of the infection (Mysore et al., 2000). The compound is in clinical trials.

Krivan et al. U.S. Pat. No. 5,446,681 (November 1995) describes bacterium receptor antibiotic conjugates comprising an asialo ganglioside coupled to a penicillin antibiotic. Especially is claimed the treatment of Helicobacter pylori with the amoxicillin-asialo-GM1 conjugate. The oligosaccharide sequences/glycolipids described by the invention do not belong to the ganglioseries of glycolipids.

US patents of Krivan et al.: U.S. Pat. No. 5,386,027 (January 1995) and U.S. Pat. No. 5,217,715 (June 1993) describe use of oligosaccharide sequences or glycolipids to inhibit several pathogenic bacteria, however the current binding specificity is not included and Helicobacter pylori is not among the bacteria studied or claimed.

The saccharide sequence GlcNAcβ3Gal has been described as a receptor for Streptococcus (Andersson et al., 1986). Some bacteria may have overlapping binding specificities, but it is not possible to predict the bindings of even closely related bacterial adhesins. In case of Helicobacter pylori the saccharide binding molecules, except the Lewis b binding protein are not known.

### SUMMARY OF THE INVENTION

The present invention relates to use of a substance or receptor binding to Helicobacter pylori comprising the oligosaccharide sequence

[Gal(A)_{q}(NAc)ᵣ/Glc(A)_{q}(NAc)ᵣα3/β3]ₛ [Galβ4GlcNAcβ3]ₜ Galβ4Glc(NAc)ᵤ

wherein q, r, s, t, and u are each independently 0 or 1,

so that when t=0 and u=0, then the oligosaccharide sequence is linked to a polyvalent carrier or present as a free oligosaccharide in high concentration, and analogs or derivatives of said oligosaccharide sequence having binding activity to Helicobacter pylori for the production of a composition having Helicobacter pylori binding or inhibiting activity.

Among the objects of the invention are the use of the Helicobacter pylori binding oligosaccharide sequences described in the invention as a medicament, and the use of the same for the manufacture of a pharmaceutical composition, particularly for the treatment of any condition due to the presence of Helicobacter pylori.

The present invention also relates to the methods for the treatment of conditions due to the presence of Helicobacter pylori. The invention is also directed to the use of the receptor(s) described in the invention as Helicobacter pylori binding or inhibiting substance for diagnostics of Helicobacter pylori.

Another object of the invention is to provide substances, pharmaceutical compositions and nutritional additives or compositions containing Helicobacter pylori binding oligosaccharide sequence(s).

Other objects of the invention are the use of the above-mentioned Helicobacter pylori binding substances for the identification of bacterial adhesin, the typing of Helicobacter pylori, and the Helicobacter pylori binding assays.

Yet another object of the invention is the use of the above-mentioned Helicobacter pylori binding substances for the production of a vaccine against Helicobacter pylori.

### BRIEF DESCRIPTION OF THE DRAWINGS

FIGS. 1A and 1B. EI/MS of permethylated oligosaccharides obtained from hexaglycosylceramide by endoglycoceramidase digestion. Gas chromatogram of the oligosaccharides (top) and EI/MS spectra of peaks A and B, respectively (bottom).

FIGS. 2A and 2B. Negative-ion FAB mass spectra of hexa-(2A) and pentaglycosylceramide (2B).

FIGS. 3A and 3B. Proton NMR spectra showing the anomeric region of the six-sugar glycolipid (3A) and the five-sugar glycolipid (3B). Spectra were acquired overnight to get good signal-to-noise for the minor type 1 component.

FIGS. 4A, 4B and 4C. Enzymatic degradation of rabbit thymus glycosphingolipids. Silica gel thin layer plates were developed in C/M/H₂O, (60:35:8, by vol.). 4A and 4B, 4-methoxybenzaldehyde visualized plates. 4C, autoradiogram after overlay with ³⁵S-labeled Helicobacter pylori. 1, heptaglycosylceramide (structure 1, Table I); 2, desialylated heptaglycosylceramide (obtained after acid treatmet); 3, desialylated heptaglycosylceramide treated with β4-galactosidase; 4, heptaglycosylceramide treated with sialidase and β4galactosidase; 5, reference glycosphingolipids from human erythrocytes (lactosylceramide, trihexosylceramide and globoside); 6, desialylated heptaglycosylceramide treated with β4-galactosidase and β-hexosaminidase; 7, heptaglycosylceramide treated with sialidase, β4-galactosidase and β-hexosaminidase.

FIGS. 5A and 5B. TLC of products obtained after partial acid hydrolysis of rabbit thymus heptaglycosylceramide (structure 1, Table I). Developing solvent was as for FIGS. 4A, 4B and 4C. 5A, 4-methoxybenzaldehyde-visualized plate; 5B, autoradiogram after overlay with ³⁵S-labeled Helicobacter pylori. 1, heptaglycosylceramide; 2, desialylated heptaglycosylceramide (acid treatment); 3, pentaglycosylceramide; 4, hydrolysate; 5, reference glycosphingolipids (as for FIGS. 4A, 4B and 4C).

FIGS. 6A and 6B. Dilution series of glycosphingolipids. The binding activity on TLC plates was determined using bacterial overlay technique. TLC developing solvent was as for FIGS. 4A, 4B and 4C. Different glycolipids were applied to the plates in equimolar amounts. Quantification of the glycolipids was based on hexose content. 6A, hexa- and pentaglycosylceramides (structures 2 and 3, Table I); 6B, penta- and tetraglycosylceramides (structures 4 and 5, Table I). The amounts of glycolipids (expressed as pmols) were as follows: 1, 1280 (of each); 2, 640; 3, 320; 4, 160; 5, 80; 6, 40; 7, 20 pmols (of each).

FIGS. 7A and 7B. Thin-layer chromatogram with separated glycosphingolipids detected with 4-methoxybenzaldehyde (7A) and autoradiogram after binding of radiolabeled Helicobacter pylori strain 032 (7B). The glycosphingolipids were separated on aluminum-backed silica gel 60 HPTLC plates (Merck) using chloroform/methanol/water 60:35:8 (by volume) as solvent system. The binding assay was done as described in the “Materials and methods” section. Autoradiography was for 72 h. The lanes contained:

lane 1) Galβ4GlcNAcβ3Galβ4Glcβ1Cer (neolactotetraosylceramide), 4 μg;

lane 2) Galα3Galβ4GlcNAcβ3Galβ4Glcβ1Cer (B5 glycosphingolipid), 4 μg;

lane 3) Galα3Galβ4GlcNH₂β3Galβ4Glcβ1Cer, 4 μg;

lane 4) Galα3(Fucα2)Galβ4GlcNAcβ3Galβ4Glcβ1Cer (B6 type 2 glycosphingolipid), 4 μg;

lane 5) GlcNAcβ3Galβ4GlcNAcβ3Galβ4Glcβ1Cer, 4 μg;

lane 6) Galβ4GlcNAcβ3Galβ4GlcNAcβ3Galβ4Glcβ1Cer, 4 μg;

lane 7) GalNAcβ3Galβ4GlcNAcβ3Galβ4Glcβ1Cer (x₂ glycosphingolipid), 4 μg;

lane 8) NeuAcα3GalNAcβ3Galβ4GlcNAcβ3Galβ4Glcβ1Cer (NeuAc-x₂), 4 μg;

lane 9) Fucα2Galβ4GlcNAcβ3Galβ4Glcβ1Cer (H5 type 2 glycosphingolipid), 4 μg;

lane 10) NeuAcα3Galβ4GlcNAcβ3Galβ4Glcβ1Cer

(sialylneolactotetraosylceramide), 4 μg. The sources of the glycosphingolipids are the same as given in Table 2.

FIGS. 8A, 8B, 8C and 8D. Calculated minimum energy conformations of three glycosphingolipids which bind Helicobacter pylori: GalNAcβ3Galβ4GlcNAcβ3Galβ4GlcβCer (8A), GalNAcα3Galβ4GlcNAcβ3Galβ4GlcβCer (8B) and Galα3Galβ4GlcNAcβ3Galβ4GlcβCer (8C). Also shown is the non-binding Galα3Galβ4GlcNH₂βGalβ4GlcβCer structure (8D). Top views of the oligosaccharide part of each of the calculated minimum energy structures are also shown. Despite differences in anomerity, absence or presence of an acetamido group, axial or equatorial position of the 4-OH of the terminal sugar and the fact that the ring plane of the terminal α3-linked compounds is raised somwhat above the corresponding plane of the one being β3-linked, a substantial topographical similarity exists between these structures and also the GlcNAcβ3-terminated structure derived from rabbit thymus (see FIG. 9A), thus explaining their similar affinities for the bacterial adhesin. In contrast, the acetamido group of the internal GlcNAcβ3 is essential for binding (cf. 8C and 8D).

FIGS. 9A, 9B, 9C and 9D. Calculated minimum energy conformations of the binding-active glycosphingolipids GlcNAcβ3Galβ4GlcNAcβ3Galβ4GlcβCer (9A) and Galβ4GlcNAcβ3Galβ4-GlcNAcβ3Galβ4GlcβCer (9B) and the non-binding glycosphingolipids NeuAcα3GalNAcβ3Galβ4GlcNAcβ3Galβ4GlcβCer (9C) and Galα3(Fucα2)Galβ4GlcNAcβ3Galβ4Glcβ3Cer (9D). The latter two extensions (9C and 9D) abolish binding of Helicobacter pylori while the former (9B) is tolerated but results in a reduced affinity. Together with the finding that de-N-acylation of the acetamido moiety of the internal GlcNAc of B5 (FIGS. 8A, 8B, 8C and 8D) completely abolishes binding, the part constituting the binding epitope must consist of the terminal trisaccharide of B5 shown in FIG. 8C since the acetamido group of a terminally situated N-acetylgalactosamine is non-essential.

FIG. 10. Minimum energy conformer of the seven-sugar compound NeuGcα3Galβ4GlcNAcβ3Galβ4GlcNAcβ3Galβ4GlcβCer shown in two projections rotated 90 degrees relative each other. The terminal carbon atom of the glycolyl moiety of the sialic acid as well as the methyl carbon atoms of the acetamido groups of the two internal GlcNAc residues are indicated in black only in order to facilitate the viewer's orientation. For the Glcβcer linkage the extended conformation was arbitrarily chosen for presentation but the minimum binding sequence GlcNAcβ3Galβ4GlcNAcβ3 is most likely better exposed toward an approaching adhesin in GlcβCer conformations other than the one shown here.

FIGS. 11A, 11B and 11C. Binding of the monoclonal antibody TH2 (11B) and the lectin from E. cristagalli (11C) to total non-acid glycosphingolipid fractions from epithelial cells from human gastric mucosa, human granulocytes and human erythrocytes separated on thin-layer chromatograms. In (11A) the same fractions are shown with 4-methoxybenzaldehyde staining. Autoradiography was in cases (11B) and (11C) performed for twelve hours. In lanes 1-6 80 μg of the total non-acid fractions from epithelial cells from human gastric mucosa of five different blood group A individuals were applied, whereas in lane 6 40 μg from the total non-acid fraction from human granulocytes and in lane 7 40 μg from the total non-acid fraction from human erythrocytes were applied. The overlay assays were performed as described in “Materials and methods”.

### DETAILED DESCRIPTION OF THE INVENTION

The present invention describes a family of specific oligosaccharide sequences binding to Helicobacter pylori. Numerous naturally occuring glycosphingolipids were screened by thin-layer overlay assay (Table 2). The structures of the glycosphingolipids used were characterized by proton NMR and mass spectrometric experiments. Molecular modeling was used to compare three dimensional structures of the substances binding to Helicobacter pylori.

The novel binding specificity was demonstrated by comparing four pentasaccharide glycolipids. It was found that the exchange of the non-reducing end terminal saccharide in GlcNAcβ3Galβ4GlcNAcβ3Galβ4GlcβCer by either GalNAcβ3 (short name x₂ GSL), GalNAcα3 or Galα3 (B5) all resulted in binding of Helicobacter pylori, despite differences in anomerity, absence or presence of an acetamido moiety and axial/equatorial position of the 4-OH. The specificity also includes structures with weaker binding to Helicobacter pylori: a shorter form Galβ4GlcNAcβ3Galβ4GlcβCer and β4-elongated forms of the glycolipid with terminal N-acetylglucosamine: Galβ4GlcNAcβ3Galβ4GlcNAcβ3Galβ4GlcβCer and NeuGcα3Galβ4GlcNAcβ3Galβ4GlcNAcβ3Galβ4GlcβCer. In contrast to previously known sialic acid depending specificities (Evans et al., 1988; Miller-Podraza et al., 1996; 1997a), the N-glycolyl neuraminic acid of the last mentioned glycosphingolipid could be released without effect to the binding of Helicobacter pylori.

The binding to GlcNAcβ3Galβ4GlcNAcβ3Galβ4GlcβCer was very reproducible, though the general saccharide bindings of Helicobacter pylori suffer from phase variations of the bacterium, and high affinity of the binding was visible in the overlay assay at low picomolar amounts of the glycolipid.

The length of the binding epitope was indicated by experiments showing that GlcNAcβ3Galβ4GlcβCer, Galβ4GlcNβ3Galβ4GlcβCer, and Galα3Galβ4GlcNβ3Galβ4GlcβCer (a shortened form and N-deacetylated forms of the active species) were not binding to Helicobacter pylori. The data reveal that the inner GlcNAc residue participites in binding but does not create strong enough binding alone. The binding epitope was considered to be the terminal trisaccharide in the pentasaccharide epitopes discussed above. When only two of the residues are present as in Galβ4GlcNAcβ3Galβ4GlcβCer, binding is weaker, and in the hexasaccharide glycolipid Galβ4GlcNAcβ3Galβ4GlcNAcβ3Galβ4GlcβCer the terminal Galβ4 inhibits the binding, explaining the weaker activity. A heptasaccharide glycolipid having Galα3 on the less active hexasaccharide glycolipid strucure, Galα3Galβ4GlcNAcβ3Galβ4GlcNAcβ3Galβ4GlcβCer, had higher activity also indicating that terminal trisaccharide epitopes are required for good binding activity.

Specificity of the binding was characterized by assaying isomers and modified forms of the active species. Elongated forms of Galβ4GlcNAcβ3Galβ4GlcβCer having the following modifications on the terminal Gal: Fucα2 (short name HS-2), Fucα2 and Gal/GalNAcα3 (B6-2, A6-2), Neu5Acα3 or Neu5Acα6 (sialylparaglobosides), or Galα4 (P₁) were inactive in the binding assays with Helicobacter pylori. The binding was also destroyed by having a 6-linked branch inner galactose, shown by the structure Galβ4GlcNAcβ3(Galβ4GlcNAcβ6)Galβ4GlcβCer. The branch has been shown to change the presentation of the Galβ4GlcNAcβ3-epitope and the disaccharide binding site is probably sterically hindered (Teneberg et al., 1994). (However the result shows that the inner galactose residue to which the disaccharide- or trisaccharide binding epitopes are bound by P3-linkage may also contribute to binding.) Furthermore Neu5Acα3GalNAcβ3Galβ4GlcNAcβ3Galβ4GlcβCer (an elongated form of the binding active x₂-glycosphingolipid) or GalNAcβ3Galβ3Galβ4GlcNAcβ3Galβ4Glcβ3Cer (elongated B5 GSL) did not appear to bind to Helicobacter pylori.

Molecular modeling was used to compare the active binding structures and inactive species. Calculated minimum energy conformers of the four pentasaccharide glycosphingolipids (Galβ4GlcNAcβ3Galβ4GlcβCer with elongation by either GlcNAcβ3, GaNAcβ3, GalNAcα3 or Galα3) show that conformations of the compounds may closely mimic each other. The conformations of the inactive glycolipids were different. Despite the fact that the terminal saccharides differ also in their-anomeric linkage (two alfa- and two beta-linked), molecular modeling revealed that the minimum energy structures are topographically very similar. The differences of the terminal structures are that Galα3 lacks an acetamido group present in the other three, Gal and GalNAc have the 4-OH in the axial position and GlcNAc in the equivatorial position, and the ring planes of the alfa anomeric terminal are raised slightly above the corresponding plane in the beta anomeric ones. The elongation of the terminal is allowed on position 4 of GlcNAc, also indicating that the 4-OH is not very important for the binding, though the Galβ4 elongation causes steric interference. In conclusion, neither the position of 4-OH nor the absence/presence of an acetamido group nor the anomeric structure of terminal monosaccharide residue appear to be crucial for binding to occur, since all the four pentasaccharide glycolipids have similar affinities for the Helicobacter pylori adhesin.

In the light of these rules of binding four other terminal monosaccharides in the binding substance may also provide trisaccharide binding epitopes: Gal(33Gal134GlcNAc, GlcNAcα3Galβ4GlcNAc, Glcβ3Galβ4GlcNAc and Glcα3Galβ4GlcNAc. These are analogous to the sequences studied only having differences in the anomeric, 4-epimeric or on C2 NAc/OH structures. The first one is present on a glycolipid from human erythrocytes, while the last three are not known from human tissues so far, but could rather represent analogues of the natural receptor.

The binding epitope was shown to include the terminal trisaccharide element of active pentasaccharide glycolipids, and at least in larger repetitive N-acetyllactosamines the epitope may be also in the middle of the saccharide chain. The inventors realize that the binding epitopes can be presented in numerous ways on natural or biosynthetically produced glycoconjugates and oligosaccharides such as O-linked or N-linked glycans of glycoproteins and on poly-N-acetyllactosamine oligosaccharides. Chemical and enzymatic synthesis methods, especially in the carbohydrate field, allow production of almost an infinite number of derivatives and analogs. The size of the binding epitope allows some modifications, as exemplified on the C1, C2 and C4 of the terminal monosaccharide, by loss of the non-reducing terminal monosaccharide or elongation from C4 of terminal GlcNAc of GlcNAcβ3Galβ4GlcNAc, e.g., the position C4 of GlcNAcβ3 can be linked to an oligosaccharide chain by a glycosidic bond. When the oligosaccharide sequence is GlcNAcβ3Galβ4GlcNAcβ3Galβ4Glc, position C4 of terminal GlcNAcβ3 can be linked to Galβ1- or an oligosaccharide chain by a glycosidic bond. Especially the C2 and C4 positions of the non-reducing terminal monosaccharide residue in the trisaccharide epitope and the reducing ends of the epitopes can be used for making derivatives and oligomeric or polymeric conjugates having binding activity to Helicobacter pylori. The C6 positions of the monosaccharide residues can also be used to produce derivatives and analogs, especially the C6 position of the non-reducing terminal residue in trisaccharide sequence and the reducing end residue of di- and trisaccharide binding substances are preferred.

In this invention the terms “analog” and “derivative” are defined as follows. According to the present invention it is possible to design structural analogs or derivatives of the Helicobacter pylori binding oligosaccharide sequences. Thus, the invention is also directed to the structural analogs of the substances according to the invention. The structural analogs according to the invention comprises the structural elements important for the binding of Helicobacter pylori to the oligosaccharide sequences. For design of effective structural analogs it is important to know the structural element important for the binding between Helicobacter pylori and the saccharides. The important structural elements are preferably not modified or these are modified by very close mimetic of the important structural element. These elements preferably include the 4, and 6-hydroxyl groups of the Galβ4 residue in the trisaccharide and disaccharide epitopes. Also the positioning of the linkages between the ring structures is an important structural element. For a high affinity binding the acetamido group or acetamido mimicking group is preferred in the position corresponding to the acetamido group of the reducing end-GlcNAc of the di- or trisaccharide epitopes. Acetamido group mimicking group may be another amide, such as alkylamido, arylamido, secondary amine, preferentially N-ethyl or N-methyl, O-acetyl, or O-alkyl for example O-ethyl or O-methyl. For high affinity binding amide derivatives from carboxylic acid group of the terminal uronic acid and analogues thereof are preferred. The activity of non-modified uronic acid is considered to rise in lower pH. The structural derivatives according to the invention are oligosaccharide sequences according to the invention modified chemically so that the binding to the Helicobacter pylori is retained or increased. According to the invention it is preferred to derivatize one or several of the hydroxyl or acetamido groups of the oligosaccharide sequences. The invention describes several positions of the molecules which could be changed when preparing the analogs or the derivatives. The hydroxyl or acetamido groups which tolerate at least certain modifications are indicated by R-groups in Formula 1.

Bulky or acidic substituents and other structures, such as monosaccharide residues, are not tolerated at least when linked in the position of the C2, C3 or C6-hydroxyls of the Galβ4GlcNAc and on C3-hydroxyl non-reducing terminal monosaccharide of the trisaccharide epitopes. Methods to produce oligosaccharide analogs for the binding of a lectin are well known. For example, numerous analogs of sialyl-Lewis x oligosaccharide has been produced, representing the active functional groups different scaffold, see page 12090 Sears and Wong 1996. Similarily analogs of heparin oligosaccharides has been produced by Sanofi corporation and sialic acid mimicking inhibitors such as Zanamivir and Tamiflu (Relenza) for the sialidase enzyme by numerous groups. Preferably the oligosaccharide analog is build on a molecule comprising at least one six- or five-membered ring structure, more preferably the analog contains at least two ring structures comprising 6 or 5 atoms. A preferred analogue type of the oligosaccharide comprise a terminal uronic acid amide or analogue linked to Galβ4GlcNAc-saccharide mimicking structure. Alternatively terminal uronic acid amide is 1-3-linked to Gal, which is linked to the GlcNAc mimicking structure. In mimicking structures monosaccharide rings may be replaced rings such as cyclohexane or cyclopentane, aromatic rings including benzene ring, heterocyclic ring structures may comprise beside oxygen for example nitrogen and sulphur atoms. To lock the active ring conformations the ring structures may be interconnected by tolerated linker groups. Typical mimetic structure may also comprise peptide analog-structures for the oligosaccharide sequence or part of it.

The effects of the active groups to binding activity are cumulative and lack of one group could be compensated by adding an active residue on the other side of the molecule. Molecular modelling, preferably by a computer can be used to produce analog structures for the Helicobacter pylori binding oligosaccharide sequences according to the invention. The results from the molecular modelling of several oligosacharide sequences are given in examples and the same or similar methods, besides NMR and X-ray crystallography methods, can be used to obtain structures for other oligosaccharide sequences according to the invention. To find analogs the oligosaccharide structures can be “docked” to the carbohydrate binding molecule(s) of H. pylori, most probably to lectins of the bacterium and possible additional binding interactions can be searched.

It is also noted that the monovalent, oligovalent or polyvalent oligosaccharides can be activated to have higher activity towards the lectins by making derivative of the oligosaccharide by combinatorial chemistry. When the library is created by substituting one or few residues in the oligosacharide sequence, it can be considered as derivative library, alternatively when the library is created from the analogs of the oligosaccharide sequences described by the invention. A combinatorial chemistry library can be built on the oligosaccharide or its precursor or on glycoconjugates according to the invention. For example, oligosaccharides with variable reducing end can be produced by so called carbohydrid technology

In a preferred embodiment a combinatorial chemistry library is conjugated to the Helicobacter pylori binding substances described by the invention. In a more preferred embodiment the library comprises at least 6 different molecules. Preferably the combinatorial chemistry modifications are produced by different amides from carboxylic acid group on R₈ according to Formula 1. Group to be modified in R₈ may be also an aldehyde or amine or another type of reactive group. Such library is preferred for use of assaying microbial binding to the oligosaccharide sequences according to the invention. Aminoacids or collections of organic amides are commercially available, which substances can be used for the synthesis of combinatorial library of uronic acid amides. A high affinity binder could be identified from the combinatorial library for example by using an inhibition assay, in which the library compounds are used to inhibit the bacterial binding to the glycolipids or glycoconjugates described by the invention. Structural analogs and derivatives preferred according to the invention can inhibit the binding of the Helicobacter pylori binding oligosaccharide sequences according to the invention to Helicobacter pylori.

Steric hindrance by the lipid part or the proximity of the silica surface probably limits the measurement of the epitope GlcNAcβ3Galβ4Glc in current TLC-assay. Using the assay activity of this sequence could not be obtained in recent study of toxin A from Clostridium difficile, which specifically recognizes the same four trisaccharide epitopes described here for Helicobacter pylori (Teneberg et al., 1996). However, the binding of Galα3Galβ4Glc to the toxin A was demonstrated by others using a large polymeric spacer modified conjugate of the saccharide (Castagliuolo et al., 1996). Also considering the contribution of the terminal monosaccharide to the binding indicates that Glc could be allowed at the reducing end of the epitope; in the non-active N-deacetylated form the positive charge of the free amine group is probably more destructive to the binding than the presence of the hydroxyl group. The trisaccharide epitopes with Glc at reducing end are considered as effective analogs of the Helicobacter pylori binding substance when present in oligovalent or more preferably in polyvalent form. One embodiment of the present invention is the saccharides with Glc at reducing end, which are used as free reducing saccharides with high concentration, preferably in the range 1-100 g/l, more preferably 1-20 g/l. It is realized that these saccharides may have minor activity in the concentration range 0.1-1 g/l.

In the following the Helicobacter pylori binding sequence is described as an oligosaccharide sequence. The oligosaccharide sequence defined here can be a part of a natural or synthetic glycoconjugate or a free oligosaccharide or a part of a free oligosaccharide. Such oligosaccharide sequences can be bonded to various monosaccharides or oligosaccharides or polysaccharides on polysaccharide chains, for example, if the saccharide sequence is expressed as part of a bacterial polysaccharide. Moreover, numerous natural modifications of monosaccharides are known as exemplified by O-acetyl or sulphated derivative of oligosaccharide sequences. The Helicobacter pylori binding substance defined here can comprise the oligosaccharide sequence described as a part of a natural or synthetic glycoconjugate or a corresponding free oligosaccharide or a part of a free oligosaccharide. The Helicobacter pylori binding substance can also comprise a mix of the Helicobacter pylori binding oligosaccharide sequences.

Several derivations of the receptor oligosaccharide sequence reduced the binding below the limit of detection in current assay, showing the specificity of the recognition. The binding data shows that if the said oligosaccharide sequences have GalNAcβ3 linked to Galα3Galβ4GlcNAc (substituted sequence: GalNAcβ3Galα3Galβ4GlcNAc), or Neu5Acα3 linked to GalNAcβ3Galβ4GlcNAc (substituted sequence: Neu5Acα3GalNAcβ3Galβ4GlcNAc) the compounds are not active. When the said oligosaccharide sequence is Galβ4GlcNAc, it is not α4-galactosylated (sequence is not Galα4Galβ4GlcNAc), α3-, or α6-sialylated (sequence is not Neu5Acα3/6Galβ4GlcNAc), α2- or α3-facosylated [said oligosaccharide sequence is not Fucα2Galβ4GlcNAc or Galβ4(Fucα3)GlcNAc or Fucα2Galβ4(Fucα3)GlcNAc, α3-fucosylation referring to fucosylation of GlcNAc residues of lactosamine forming Lewis x, Galβ4(Fucα3)GlcNAc]. Saccharides having structures where Galβ3 is linked to GlcNAcβ3 (such as Galβ3GlcNAcβ3Galβ4GlcNAc/Glc) have different conformations in comparision to the Helicobacter pylori binding substances described herein and their binding specificies have been studied separately. The Helicobacter pylori binding substances may be part of a saccharide chain or a glycoconjugate or a mixture of glycocompounds containing other known Helicobacter binding epitopes, with different saccharide sequences and conformations, such as Lewis b (Fucα2Galβ3(Fucα4)GlcNAc) or Neu5Acα3Galβ4Glc/GlcNAc. Using several binding substances together may be beneficial for therapy.

The Helicobacter pylori binding oligosaccharide sequences can be synthesized enzymatically by glycosyltransferases, or by transglycosylation catalyzed by glycosidase or transglycosidase enzymes (Ernst et al., 2000). Specifities of these enzymes and the use of co-factors can be engineered. Specific modified enzymes can be used to obtain more effective synthesis, for example, glycosynthase is modified to do transglycosylation only. Organic synthesis of the saccharides and the conjugates described herein or compounds similar to these are known (Ernst et al., 2000). Saccharide materials can be isolated from natural sources and modified chemically or enzymatically into the Helicobacter pylori binding compounds. Natural oligosaccharides can be isolated from milks produced by various ruminants. Transgenic organisms, such as cows or microbes, expressing glycosylating enzymes can be used for the production of saccharides.

The uronic acid monosaccharide residues described in the invention can be obtained by methods known in the art. For example, the hydroxyl of the 6-carbon of N-acetylglucosamine or N-acetylgalactosamines can be chemically oxidized to carboxylic acid. The oxidation can be done to a properly protected oligosaccharide or monosaccharide.

In a preferred embodiment a non-protected polymer or oligomer comprising hexoses, N-acetylhexosamines or hexosamines, wherein the linkage between the monosaccharides is not between carbon 6 atoms, is

1) oxidized to corresponding polymer of uronic acid residues, or to polymer comprising monomers of 6-aldehydomonosacharides

2) optionally derivatized from the carboxylic acid group or 6-aldehydo group, preferentially to an amide or an amine and

3) hydrolysed to the uronic acid monosaccharides or uronic acid derivative monosaccharides.

Methods to oxidize monosaccharide residues to uronic acids and to hydrolyse amine or uronic acid polymers chemically or enzymatically are well-known in the art. It is especially preferred to use the method to oligomers or polymers of cellulose, starch or other glucans with 1-2 or 1-3 or 1-4 linkages, chitin (GlcNAc polymer) or chitosan (GlcN polymer), which are commercially available in large scale or Nacetylgalactosamine/galactosamine polysaccharides (for example, ones known from a bacterial source) is oxidized to a corresponding 1-4-linked saccharide. This method can also be applied to galactan polymers. Derivatives of uronic acid can be produced also from natural polymers comprising uronic acids such as pectins or glucuronic acid containing bacterial polysaccharides including N-acetylheparin, hyaluronic and chonroitin type bacterial exopolysaccharides. This method involves

1) derivatization of the carboxylic acid groups of the polysaccharide, preferably by an amide bond and

2) hydrolysis of the polysaccharide to the uronic acid monosaccharides or uronic acid derivative monosaccharides.

Chemical and enzymatic methods are also known to oxidize primary alcohol on carbon 6 of the polysaccharide to aldehyde or to carboxylic acid. An aldehyde can be further derivatized, for example, to amine by reductive amination. Preferably terminal Gal or GalNAc is oxidized by a primary alcohol oxidizing enzyme-like galactose oxidase and can then be further derivatized, for example, by amines.

The uronic acid residues can be conjugated to disaccharides or oligosaccharides by standard methods of organic chemistry. Alternatively GlcA can be linked by a glucuronyl transferase transferring a GlcA from UDP-GlcA to terminal Lac(NAc). Monosaccharide derivatives mimicking N-acetylhexosamines could be produced from a polymer or an oligomer comprising hexosamines or other monosaccharides with free primary amine groups by method involving:

1) derivatization of the amine groups to a secondary or tertiary amine or amide

2) hydrolysing the polymer to corresponding monosaccharides.

Chitosan and oligosaccharides thereof are an example of an amine comprising polymer or oligomer.

In general the method to produce carboxylic acid containing, 6-aldehydo comprising, amine and/or amide comprising monosaccharide/monosaccharides involves following steps

1. optionally introducing an carboxylic acid or 6-aldehydo group to a carbohydrate polymer wherein primary hydroxyl is available for modification

2. derivatization of carboxylic acid groups or 6-aldehydo groups or primary amine groups of the polymer to secondary or tertiary amines or to amides, when step 1 is applied, step 2 is optional.

3. hydrolysis of the polymer to corresponding monosaccharides. The hydrolysis to monosaccharides may also be partial and produce useful disaccharide or oligosaccharide to produce analog substances. Preferably the hydrolysis produces at least 30% of monosaccharides. Methods to produce the chemical steps are known in the art. For example oxidation of the polysaccharides to corresponding monoaccharides can be performed as described by Muzzarelli et al 1999 and 2002. These methods are preferred to the use of non-protected monosaccharides, because the protection or reactive reducing ends of the monosaccharides is avoided.

In a preferred embodiment the oligosaccharide sequences comprising GlcAβ3Lac or GlcAβ3LacNAc are effectively synthesised by transglycosylation using a specific glucuronidase such as glucuronidase from bovine liver. It was realized that the enzyme can site-specifically transfer from β1-3 linkage to Galβ4GlcNAc and Galβ4Glc with unexpectedly high yields for a transglycosylation reaction. In general such selectivity and yields close 30% or more are not obtained in transglycosylation reactions.

One embodiment of the present invention is use of a substance or a receptor binding to Helicobacter pylori comprising the oligosaccharide sequence

[Gal(A)_{q}(NAc)ᵣ/Glc(A)_{q}(NAc)ᵣα3/β3]ₛ[Galβ4GlcNAcβ3 ]ₜGalβ4Glc(NAc)ᵤ

wherein q, r, s, t, and u are each independently 0 or 1,

so that when t=0 and u=0, then the oligosaccharide sequence is linked to a polyvalent carrier or present as a free oligosaccharide in high concentration, and analogs or derivatives of said oligosaccharide sequence having binding activity to Helicobacter pylori for the production of a composition having Helicobacter pylori binding or inhibiting activity.

A in the above oligosaccharide sequence indicates uronic acid of the monosaccharide residue or carbon 6 derivative of the monosaccharide residue, most preferably the derivative of carbon 6 is an amide of the uronic acid.

The following oligosaccharide sequences are among the preferable Helicobacter pylori binding substances for the uses of the invention

Galβ4GlcNAc,

GalNAcα3Galβ4GlcNAc, GalNAcβ3Galβ4GlcNAc, GlcNAcα3Galβ4GlcNAc, GlcNAcβ3Galβ4GlcNAc, Galα3Galβ4GlcNAc, Galβ3Galβ4GlcNAc, Glcα3Galβ4GlcNAc, Glcβ3Galβ4GlcNAc,

Galβ4GlcNAcβ3Galβ4GlcNAc, Galβ4GlcNAcβ3Galβ4Glc,

GalNAcα3Galβ4GlcNAcβ3Galβ4Glc, GalNAcβ3Galβ4GlcNAcβ3Galβ4Glc, GlcNAcα3Galβ4GlcNAcβ3Galβ4Glc, GlcNAcβ3Galβ4GlcNAcβ3Galβ4Glc, Galα3Galβ4GlcNAcβ3Galβ4Glc, Galβ3Galβ4GlcNAcβ3Galβ4Glc, Glcα3Galβ4GlcNAcβ3Galβ4Glc, Glcβ3Galβ4GlcNAcβ3Galβ4Glc,

GalANAcβ3Galβ4GlcNAc, GalANAcα3Galβ4GlcNAc,GalAβ3Galβ4GlcNAc, GalAα3Galβ4GlcNAc, GalANAcβ3Galβ4Glc, GalANAcα3Galβ4Glc, GalAβ3Galβ4Glc, GalAα3Galβ4Glc,

GlcANAcβ3Galβ4GlcNAc, GlcANAcα3Galβ4GlcNAc,GlcAβ3Galβ4GlcNAc, GlcAα3Galβ4GlcNAc, GlcANAcβ3Galβ4Glc, GlcANAcα3Galβ4Glc,GlcAβ3Galβ4Glc, GlcAα3Galβ4Glc,

Galβ4GlcNAcβ3Galβ4GlcNAcβ3Galβ4Glc, and reducing-end polyvalent conjugates thereof,

as well as GalNAcα3Galβ4Glc, GalNAcβ3Galβ4Glc, GlcNAcα3Galα4Glc, GlcNAcβ3Galβ4Glc, Galα3Galβ4Glc, Galβ3Galβ4Glc, Glcα3Galβ4Glc, and Glcβ3Galβ4Glc.

Another embodiment of the invention is described in Formula 1.

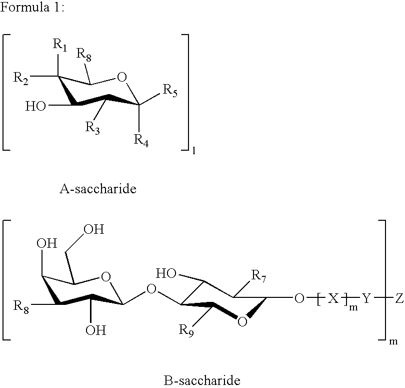

Among the preferable Helicobacter pylori binding substances or mixtures of the substances of the invention and for the uses of the invention are the oligosaccharide structures according to Formula 1, wherein integers 1, m, and n have values m≧1, 1 and n are independently 0 or 1, and wherein R₁ is H and R₂ is OH or R₁ is OH and R₂ is H or R₁ is H and R₂ is a monosaccharidyl- or oligosaccharidyl-group preferably a beta glycosidically linked galactosyl group, R₃ is independently —OH or acetamido (—NHCOCH₃) or an acetamido analogous group. R₇ is acetamido (—NHCOCH₃) or an acetamido analogous group. When 1=1, R₄ is —H and R₅ is oxygen linked to bond R₆ and forms a beta anomeric glycosidic linkage to saccharide B or R₅ is —H and R₄ is oxygen linked to bond R₆ and forms an alpha anomeric glycosidic linkage to saccharide B, when 1=0 R₆ is —OH linked to B. X is monosaccharide or oligosaccharide residue, preferably X is lactosyl-, galactosyl-, poly-N-acetyl-lactosaminyl, or part of an O-glycan or an N-glycan oligosaccharide sequence; Y is a spacer group or a terminal conjugate such as a ceramide lipid moiety or a linkage to Z. Z is an oligovalent or a polyvalent carrier. The binding substance may also be an analog or derivative of said substance according to Formula 1 having binding activity with regard to Helicobacter pylori, e.g., the oxygen linkage (—O—) between position C1 of the B saccharide and saccharide residue X or spacer group Y can be replaced by carbon (—C—), nitrogen (—N—) or sulphur (—S—) linkage.

In Formula 1 R₈ is preferably carboxylic acid amide, such as methylamide or ethyalamide, hydroxymethyl (—CH₂—OH) or a carboxylic acid group or an ester thereof, such as methyl or ethyl ester. The carboxylic acid amide may comprise an alternative linkage to the polyvalent carrier Z comprising an amine such as chitosan or galactosamine polysaccharide or Z comprising a primary amine containing spacer, preferably a hydrophilic spacer. The structure in R₈ can be also a mimicking structure known in the art to ones described above. For example secondary or tertiary amines or amidated secondary amine can be used.

In Formula 1 R₉ is preferably hydroxymethyl but it can be used for derivatisations as described for R₈.

R₃ is hydroxyl, acetamido or acetamido group mimicking group, such as C₁₋₆ alkylamides, arylamido, secondary amine, preferentially N-ethyl or N-methyl, O-acetyl, or O-alkyl for example O-ethyl or O-methyl. R₇ is same as R₃ but more preferentially acetamido or acetamido mimicking group.

R₂ may also comprise preferentially a six-membered ring structure mimicking Galβ4-terminal.

The bacterium binding substances are preferably represented in clustered form such as by glycolipids on cell membranes, micelles, liposomes, or on solid phases such as TCL-plates used in the assays. The clustered representation with correct spacing creates high affinity binding.

According to the invention it is also possible to use the Helicobacter pylori binding epitopes or naturally occurring, or a synthetically produced analogue or derivative thereof having a similar or better binding activity with regard to Helicobacter pylori. It is also possible to use a substance containing the bacterium binding substance such as a receptor active ganglioside described in the invention or an analogue or derivative thereof having a similar or better binding activity with regard to Helicobacter pylori. The bacterium binding substance may be a glycosidically linked terminal epitope of an oligosaccharide chain. Alternatively the bacterium binding epitope may be a branch of an oligosaccharide chain, preferably a polylactosamine chain.

The Helicobacter pylori binding substance may be conjugated to an antibiotic substance, preferably a penicillin type antibiotic. The Helicobacter pylori binding substance targets the antibiotic to Helicobacter pylori. Such conjugate is beneficial in treatment because a lower amount of antibiotic is needed for treatment or therapy against Helicobacter pylori, which leads to lower side effect of the antibiotic. The antibiotic part of the conjugate is aimed at killing or weaken the bacteria, but the conjugate may also have an antiadhesive effect as described below.

The bacterium binding substances, preferably in oligovalent or clustered form, can be used to treat a disease or condition caused by the presence of the Helicobacter pylori. This is done by using the Helicobacter pylori binding substances for antiadhesion, i.e. to inhibit the binding of Helicobacter pylori to the receptor epitopes of the target cells or tissues. When the Helicobacter pylori binding substance or pharmaceutical composition is administered it will compete with receptor glycoconjugates on the target cells for the binding of the bacteria. Some or all of the bacteria will then be bound to the Helicobacter pylori binding substance instead of the receptor on the target cells or tissues. The bacteria bound to the Helicobacter pylori binding substances are then removed from the patient (for example by the fluid flow in the gastrointestinal tract), resulting in reduced effects of the bacteria on the health of the patient. Preferably the substance used is a soluble composition comprising the Helicobacter pylori binding substances. The substance can be attached to a carrier substance which is preferably not a protein. When using a carrier molecule several molecules of the Helicobacter pylori binding substance can be attached to one carrier and inhibitory efficiency is improved.

The target cells are primarily epithelial cells of the target tissue, especially the gastrointestinal tract, other potential target tissues are for example liver and pancreas. Glycosylation of the target tissue may change because of infection by a pathogen (Karlsson et al., 2000). Target cells may also be malignant, transformed or cancer/tumour cells in the target tissue. Transformed cells and tissues express altered types of glycosylation and may provide receptors to bacteria. Binding of lectins or saccharides (carbohydrate-carbohydrate interaction) to saccharides on glycoprotein or glycolipid receptors can activate cells, in case of cancer/malignant cells this may be lead to growth or metastasis of the cancer. Several of the oligosaecharide epitopes described herein, such as GlcNAcβ3Galβ4GlcNAc (Hu, J. et al., 1994), Galα3Galβ4GlcNAc (Castronovo et al., 1989), and neutral and sialylated polylactosamines from malignant cells (Stroud et al., 1996), have been reported to be cancer-associated or cancer antigens. Oligosaccharide chains containing substances described herein have also been described from lymphocytes (Vivier et al, 1993). Helicobacter pylori is associated with gastric lymphoma. The substances described herein can be used to prevent binding ofHelicobacter pylori to premalignant or malignant cells and activation of cancer development or metastasis. Inhibition of the binding may cure gastric cancer, especially lymphoma. The Helicobacter pylori binding oligosaccharide sequence has been reported in the structure GlcNAcβ3Galβ4GlcNAcβ6GalNAc from human gastric mucins. This mucin epitope and similar O-glycan glycoforms are most probably natural high affinity receptors for Helicobacter pylori in human stomach. This was also indicated by high affinity binding of an analogous sequence GlcNAcβ3Galβ4GlcNAcβ6GlcNAc as neoglycolipid to Helicobacter pylori and that the sequence GlcNAcβ3Galβ4GlcNAcβ6Gal has also some binding activity towards Helicobacter pylori in the same assay. Therefore the preferred oligosaccharide sequences includes O-glycans and analogues of O-glycan sequences such as GlcNAcβ3Galβ4GlcNAcβ6GlcNAc/GalNAc/Gal, GlcNAcβ3Galβ4GlcNAcβ6GlcNAc/GalNAc/GalαSer/Thr, GlcNAcβ3Galβ4GlcNAcβ6(Gal/GlcNAcβ3)GlcNAc/GalNAc/GalαSer/Thr and glycopeptides and glycopeptide analogs comprising the O-glycan sequences. Even sequences lacking the non-reducing end GlcNAc may have some activity. Based on this all the other Helicobacter pylori binding oligosaccharide sequences (OS) and especially the trisaccharide epitopes are also especially preferred when linked from the reducing end to form structures OSβ6Gal(NAc)₀₋₁ or OSβ6Glc(NAc)₀₋₁ or OSβ6Gal(NAc)₀₋₁αSer/Thr or OSβ6Glc(NAc)₀₁₋αSer/Thr. The Ser or Thr-compounds or analogue thereof or the reducing oligosaccharides are also preferred when linked to polyvalent carrier. The reducing oligosaccharides can be reductively linked to the polyvalent carrier.

Target cells also includes blood cells, especially leukocytes. It is known that Helicobacter pylori strains associated with peptic ulcer, as the strain mainly used here, stimulates an inflammatory response from granulocytes, even when the bacteria are nonopsonized (Rautelin et al., 1994a,b). The initial event in the phagocytosis of the bacterium most likely involves specific lectin-like interactions resulting in the agglutination of the granulocytes (Ofek and Sharon, 1988). Subsequent to the phagocytotic event oxidative burst reactions occur which may be of consequence for the pathogenesis of Helicobacter pylori-associated diseases (Babior, 1978). Several sialylated and non-acid glycosphingolipids having repeating N-acetyllactosamine units have been isolated and characterized from granulocytes (Fukuda et al., 1985; Stroud et al., 1996) and may thus act as potential receptors for Helicobacter pylori on the white blood cell surface. Furthermore, also the X₂ glycosphingolipid has been isolated from the same source (Teneberg, S., unpublished). The present invention confirms the presence of receptor saccharides on human erythrocytes and granulocytes which can be recognized by an N-acetyllactosamine specific lectin and by a monoclonal antibody (X₂, GalNAcβ3Galβ4GlcNAc-). The Helicobacter pylori binding substances can be useful to inhibit the binding of leukocytes to Helicobacter pylori and in prevention of the oxidative burst and/or inflammation following the activation of leukocytes.

It is known that Helicobacter pylori can bind several kinds of oligosaccharide sequences. Some of the binding by specific strains may represent more symbiotic interactions which do not lead to cancer or severe conditions. The present data about binding to cancer-type saccharide epitopes indicates that the Helicobacter pylori binding substance can prevent more pathologic interactions, in doing this it may leave some of the less pathogenic Helicobacter pylori bacteria/strains binding to other receptor structures. Therefore total removal of the bacteria may not be necessary for the prevention of the diseases related to Helicobacter pylori. The less pathogenic bacteria may even have a probiotic effect in the prevention of more pathogenic strains of Helicobacter pylori.

It is also realized that Helicobacter pylori contains large polylactosamine oligosaccharides on its surface which at least in some strains contains non-fucosylated epitopes which can be bound by the bacterium as described by the invention. The substance described herein can also prevent the binding between Helicobacter pylori bacteria and that way inhibit bacteria for example in process of colonization.

According to the invention it is possible to incorporate the Helicobacter pylori binding substance, optionally with a carrier, in a pharmaceutical composition, which is suitable for the treatment of a condition due to the presence of Helicobacter pylori in a patient or to use the Helicobacter pylori binding substance in a method for treatment of such conditions. Examples of conditions treatable according to the invention are chronic superficial gastritis, gastric ulcer, duodenal ulcer, non-Hodgkin lymphoma in human stomach, gastric adenocarcinoma, and certain pancreatic, skin, liver, or heart diseases, sudden infant death syndrome, autoimmune diseases including autoimmune gastritis and pernicious anaemia and non-steroid anti-inflammatory drug (NSAID) related gastric disease, all, at least partially, caused by the Helicobacter pylori infection.

The pharmaceutical composition containing the Helicobacter pylori binding substance may also comprise other substances, such as an inert vehicle, or pharmaceutically acceptable carriers, preservatives etc, which are well known to persons skilled in the art. The Helicobacter pylori binding substance can be administered together with other drugs such as antibiotics used against Helicobacter pylori.

The Helicobacter pylori binding substance or pharmaceutical composition containing such substance may be administered in any suitable way, although an oral administration is preferred.

The term “treatment” used herein relates both to treatment in order to cure or alleviate a disease or a condition, and to treatment in order to prevent the development of a disease or a condition. The treatment may be either performed in a acute or in a chronic way.

The term “patient”, as used herein, relates to any human or non-human mammal in need of treatment according to the invention.

It is also possible to use the Helicobacter pylori binding substance to identify one or more adhesins by screening for proteins or carbohydrates (by carbohydrate-carbohydrate interactions) that bind to the Helicobacter pylori binding substance. The carbohydrate binding protein may be a lectin or a carbohydrate binding enzyme.

The screening can be done for example by affinity chromatography or affinity cross lining methods (Ilver et al., 1998).

Furthermore, it is possible to use substances specifically binding or inactivating the Helicobacter pylori binding substances present on human tissues and thus prevent the binding of Helicobacter pylori. Examples of such substances include plant lectins such as Erythrina cristagalli and Erythrina corallodendron (Teneberg et al., 1994). When used in humans, the binding substance should be suitable for such use such as a humanized antibody or a recombinant glycosidase of human origin which is non-immunogenic and capable of cleaving the terminal monosaccharide residue/residues from the Helicobacter pylori binding substances. However, in the gastrointestinal tract, many naturally occuring lectins and glycosidases originating for example from food are tolerated.

Furthermore, it is possible to use the Helicobacter pylori binding substance as part of a nutritional composition including food- and feedstuff. It is preferred to use the Helicobacter pylori binding substance as a part of so called functional or functionalized food. The said functional food has a positive effect on the person's or animal's health by inhibiting or preventing the binding of Helicobacter pylori to target cells or tissues. The Helicobacter pylori binding substance can be a part of a defined food or functional food composition. The functional food can contain other acceptable food ingredients accepted by authorities such as Food and Drug Administration in the USA. The Helicobacter pylori binding substance can also be used as a nutritional additive, preferably as a food or a beverage additive to produce a functional food or a functional beverage. The food or food additive can also be produced by having, e.g., a domestic animal such as a cow or other animal produce the Helicobacter pylori binding substance in larger amounts naturally in its milk. This can be accomplished by having the animal overexpress suitable glycosyltransferases in its milk. A specific strain or species of a domestic animal can be chosen and bred for larger production of the Helicobacter pylori binding substance. The Helicobacter pylori binding substance for a nutritional composition or nutritional additive can also be produced by a micro-organisms such as a bacteria or a yeast.

It is especially useful to have the Helicobacter pylori binding substance as part of a food for an infant, preferably as a part of an infant formula. Many infants are fed by special formulas in replacement of natural human milk. The formulas may lack the special lactose based oligosaccharides of human milk, especially the elongated ones such as lacto-N-neotetraose, Galβ4GlcNAcβ3Galβ4Glc, and its derivatives. The lacto-N-neotetraose and para-lacto-N-neohexaose (Galβ4GlcNAcβ3Galβ4GlcNAcβ3Galβ4Glc) as well as Galβ3Galβ4Glc are known from human milk and can therefore be considered as safe additives or ingredients in an infant food. Helicobacter pylori is especially infective with regard to infants or young children, and considering the diseases it may later cause it is reasonable to prevent the infection. Helicobacter pylori is also known to cause sudden infant death syndrome, but the strong antiobiotic treatments used to eradicate the bacterium may be especially unsuitable for young children or infants.

Preferred concentrations for human milk oligosaccharides in functional food to be consumed (for example, in reconstituted infant formula) are similar to those present in natural human milk. It is noted that natural human milk contains numerous free oligosaccharides and glycoconjugates (which may be polyvalent) comprising the oligosaccharide sequence(s) described by the invention, wherefore it is possible to use even higher than natural concentrations of single molecules to get stronger inhibitory effect against Helicobacter pylori without harmful side effects. Natural human milk contains lacto-N-neotetraose at least in range about 10-210 mg/l with individual variations (Nakhla et al., 1999). Consequently, lacto-N-neotetraose is preferably used in functional food in concentration range 0.01-10 g/l, more preferably 0.01-5 g/l, most preferably 0.1-1 g/l. When the free oligosaccharides described herein are trisaccharides or the disaccharide with sequence Galβ4Glc at the reducing end, they are preferably consumed in concentrations 1-100 g/l, more preferably in the concentration range 1-20 g/l. Alternatively, the total concentration of the saccharides used in functional food is the same or similar to the total concentration of natural human milk saccharides, which bind Helicobacter pylori like the substances described, or which contain the binding epitope/oligosaccharide sequence indicated in the invention. At least in one case human milk has been reported to contain Galβ3Galβ4Glc as a major neutral oligosaccharide with high concentration (Charlwood et al., 1999).

Furthermore, it is possible to use the Helicobacter pylori binding substance in the diagnosis of a condition caused by an Helicobacter pylori infection. Diagnostic uses also include the use of the Helicobacter pylori binding substance for typing of Helicobacter pylori. When the substance is used for diagnosis or typing, it may be included in, e.g., a probe or a test stick, optionally constituting a part of a test kit. When this probe or test stick is brought into contact with a sample containing Helicobacter pylori, the bacteria will bind the probe or test stick and can be thus removed from the sample and further analyzed.

The results also show that the non-reducing end terminal monosaccharide residue in the preferred trisaccharide sequences of the invention can contain a carboxylic acid group on the carbon 6 (terminal monosaccahride residue is a uronic acid, HexA or HexANAc, wherein Hex is Gal or Glc) or a derivative of the carbon 6 of the HexA(NAc) residue or a derivative of the carbon 6 of the corresponding Hex(NAc) residue. Such terminal residues includes preferably β3-linked glucuronic acid and more preferably 6-amides such as methylamide thereof. Therefore analogs and derivatives of the sequence can be produced by changing or derivatising the terminal 6-position of the trisaccharide epitopes.

Preferred Helicobacter pylori Binding Substances

The oligosaccharide sequences according to the invention were found to be unexpectedly effective binders when presented on thin layer surface. This method allows polyvalent presentation of the glycolipid sequences. The surprisingly high activity of the polyvalent presentation of the oligosaccharide sequences makes polyvalency a preferred way to represent the oligosaccharide sequences of the invention.

The glycolipid structures are naturally presented in a polyvalent form on cellular membranes. This type of representation can be mimicked by the solid phase assay described below or by making liposomes of glycolipids or neoglycolipids.

The present novel neoglycolipids produced by reductive amination of hydrophobic hexadecylaniline were able to provide effective presentation of the oligosaccharides. Most previously known neoglycolipid conjugates used for binding of bacteria have contained a negatively charged groups such as phosphor ester of phosphadityl ethanolamine neoglycolipids. Problems of such compounds are negative charge of the substance and natural biological binding involving the phospholipid structure. Negatively charged molecules are known to be involved in numerous non-specific bindings with proteins and other biological substances. Moreover, many of these structures are labile and can be enzymatically or chemically degraded. The present invention is directed to the non-acidic conjugates of oligosaccharide sequences meaning that the oligosaccharide sequences are linked to non-acidic chemical structures. Preferably, the non-acidic conjugates are neutral meaning that the oligosaccharide sequences are linked to neutral, non-charged, chemical structures. The preferred conjugates according to the invention are polyvalent substances.

In the previous art bioactive oligosaccharide sequences are often linked to carrier structures by reducing a part of the receptor active oligosaccharide structure. Hydrophobic spacers containing alkyl chains (—CH₂—)ₙ and/or benzyl rings have been used. However, hydrophobic structures are in general known to be involved in non-specific interactions with proteins and other bioactive molecules.

The neoglycolipid data of the examples below show that under the experimental conditions used in the assay the hexadecylaniline parts of the neoglycolipid compounds do not cause non-specific binding for the studied bacterium. In the neoglycolipids the hexadecylaniline part of the conjugate forms probably a lipid layer like structure and is not available for the binding. The invention shows that reducing a monosaccharide residue belonging to the binding epitope may destroy the binding. It was further realized that a reduced monosaccharide can be used as a hydrophilic spacer to link a receptor epitope and a polyvalent presentation structure. According to the invention it is preferred to link the bioactive oligosaccharide via a hydrophilic spacer to a polyvalent or multivalent carrier molecule to form a polyvalent or oligovalent/multivalent structure. All polyvalent (comprising more than 10 oligosaccharide residues) and oligovalent/multivalent structures (comprising 2-10 oligosaccharide residues) are referred here as polyvalent structures, though depending on the application oligovalent/multivalent constructs can be more preferred than larger polyvalent structures. The hydrophilic spacer group comprises preferably at least one hydroxyl group. More preferably the spacer comprises at least two hydroxyl groups and most preferably the spacer comprises at least three hydroxyl groups.

According to the invention the hydrophilic spacer group is preferably a flexible chain comprising one or several —CHOH— groups and/or an amide side chain such as an acetamido —NHCOCH₃ or an alkylamido. The hydroxyl groups and/or the acetamido group also protects the spacer from enzymatic hydrolysis in vivo. The term flexible means that the spacer comprises flexible bonds and do not form a ring structure without flexibility. A reduced monosaccharide residues such as ones formed by reductive amination in the present invention are examples of flexible hydrophilic spacers. The flexible hydrophilic spacer is optimal for avoiding non-specific binding of neoglycolipid or polyvalent conjugates. This is essential optimal activity in bioassays and for bioactivity of pharmaceuticals or functional foods, for example.

A general formula for a conjugate with a flexible hydrophilic linker has the following Formula 2:

[OS—O—(X)ₙ-L₁-CH(H/{CH₁₋₂OH}ₚ₁)—{CH₁OH}ₚ₂—{CH(NH—R)}ₚ₃—{CH₁OH}ₚ₄-L₂]ₘ-Z

wherein L₁ and L₂ are linking groups comprising independently oxygen, nitrogen, sulphur or carbon linkage atom or two linking atoms of the group forming linkages such as —O—, —S—, —CH₂—, —N—, —N(COCH3)-, amide groups —CO—NH— or —NH—CO— or —N—N— (hydrazine derivative) or amino oxy-linkages —O—N— and —N—O—. L1 is linkage from carbon 1 of the reducing end monosaccharide of X or when n=0, L1 replaces —O— and links directly from the reducing end C1 of OS.

p1, p2, p3, and p4 are independently integers from 0-7, with the proviso that at least one of p1, p2, p3, and p4 is at least 1. CH₁₋₂OH in the branching term {CH₁₋₂OH}ₚ₁ means that the chain terminating group is CH₂OH and when the p1 is more than 1 there is secondary alcohol groups —CHOH— linking the terminating group to the rest of the spacer. R is preferably acetyl group (—COCH₃) or R is an alternative linkage to Z and then L₂ is one or two atom chain terminating group, in another embodiment R is an analog forming group comprising C₁₋₄ acyl group (preferably hydrophilic such as hydroxy alkyl) comprising amido structure or H or C₁₋₄ alkyl forming an amine. And m>1 and Z is polyvalent carrier. OS and X are defined in Formula 1.

Preferred polyvalent structures comprising a flexible hydrophilic spacer according to formula 2 include Helicobacter pylori binding oligosaccharide sequence(OS) β1-3 linked to Galβ4Glc(red)-Z, and OSβ6GlcNAc(red)-Z and OSβ6GalNAc(red)-Z., where “(red)” means the amine linkage structure formed by reductive amination from the reducing end monosaccharides and an amine group of the polyvalent carrier Z.

In the present invention the oligosaccharide group is preferably linked in a polyvalent or an oligovalent form to a carrier which is not a protein or peptide to avoid antigenicity and possible allergic reactions, preferably the backbone is a natural non-antigenic polysaccharide.

When the binding activities of glycolipids and neoglycolipids were compared, the sequences with Galα3Galβ- were found to have lower activity in the polyvalent presentation on thin layer plate. The sequences with terminal Galβ4GlcNAc-sequence were also weaker. Therefore the optimal polyvalent non-acidic substance according to the invention comprises a terminal oligosaccharide sequence

Gal(A)_{q1}(NAC)ᵣ₁/Glc(A)_{q2}(NAc)ᵣ₂α3/β3Galβ4Gc(NAc)ᵤ

wherein q1, q2, r1, r2, and u are each independently 0 or 1, with the proviso that when both q1 and r1 are 0, then the non-reducing end terminal monosaccharide residue is not Galα. More preferably u=0 and most preferably the oligosaccharide sequence presented in polyvalent form is

GalNAc/Glc(NAc)ᵣ₂α3/β3Galβ4GlcNAc

wherein r2 is independently 0 or 1 and an analog or derivative thereof.

Following oligosaccharide sequences are especially preferred. These represent structures, which have not been described from human or animal tissues:

Glc(A)_{q}(NAc)ᵣα3/β3Galβ4Glc(NAc)ᵤ

with the proviso that when the oligosaccharide sequence contains β3 linkage, q and r are 1 or 0; or GalA(NAc)ᵣα3/β3Galβ4Glc(NAc)ᵤ.

The novelty of the above oligosaccharide sequences makes them especially preferred. There are no known glycosidases cleaving such sequences. Therefore, the sequences are especially stable and preferred under biolological conditions. The natural type of the sequences described by the invention can be cleaved by glycosidase enzymes which reduces usefulness of these especially when used in human and animal body. Glycosidase enzymes cleaving the sequences are known to be active in human gastrointestinal tract. Several glycosidases such as N-acetylhexosaminidases or galactosidases has been described as digestive enzyme and are also present in food stuffs.

It is realized that the novel substances according to the invention are also useful for inhibiting toxin A of Clostridium difficile S. Teneberg et al 1996. The binding profile of the toxin A with older substances is very similar to specificity of Helicobacter pylori described here. Thus, the Helicobacter pylori binding sustances may be used for the treatment, for example, Clostridium difficile dependent diarrhea.

Glycolipid and carbohydrate nomenclature is according to recommendations by the IUPAC-IUB Commission on Biochemical Nomenclature (Carbohydrate Res. 1998, 312, 167; Carbohydrate Res. 1997, 297, 1; Eur. J. Biochem. 1998, 257, 29).

It is assumed that Gal, Glc, GlcNAc, and Neu5Ac are of the D-configuration, Fuc of the L-configuration, and all the monosaccharide units in the pyranose form. Glucosamine is referred as GlcN or GlcNH₂ and galactosamine as GalN or GalNH₂. Glycosidic linkages are shown partly in shorter and partly in longer nomenclature, the linkages of the Neu5Ac-residues α3 and α6 mean the same as α2-3 and α2-6, respectively, and with other monosaccharide residues α1-3, β1-3, β1-4, and β1-6 can be shortened as α3, β3, β4, and β6, respectively. Lactosamine refers to N-acetyllactosamine, Galβ4GlcNAc, and sialic acid is N-acetylneuraminic acid (Neu5Ac) or N-glycolylneuraminic acid (Neu5Gc) or any other natural sialic acid. Term glycan means here broadly oligosaccharide or polysaccharide chains present in human or animal glycoconjugates, especially on glycolipids or glycoproteins. In the shorthand nomenclature for fatty acids and bases, the number before the colon refers to the carbon chain lenght and the number after the colon gives the total number of double bonds in the hydrocarbon chain. Abbreviation GSL refers to glycosphingolipid. Abbreviations or short names or symbols of glycosphingolipids are given in the text and in Tables 1 and 2. Helicobacter pylori refers also to the bacteria similar to Helicobacter pylori.

In the present invention hex(NAc)-uronic acid and their derivatives and residues are indicated as follows: GlcA is glucuronic acid and derivatives of carbon 6 of glucose or glucuronic acid, GalA is galacturonic acid and derivatives of carbon 6 of galactose or galacturonic acid, GlcANAc is N-acetylglucuronic acid and derivatives of carbon 6 of N-acetylglucosamine or is N-acetylglucosainne uronic acid and GalANAc is N-acetylgalactosamine uronic acid and derivatives of carbon 6 of N-acetylgalactosamine or N-acetylgalactosamine uronic acid.

The expression “terminal oligosaccharide sequence” indicates that the oligosaccharide is not substituted to the non-reducing end terminal residue by another monosaccharide residue.

The term “α3/β3” indicates that the adjacent residues in an oligosaccharide sequence can be either α3- or β3-linked to each other.

The present invention is further illustrated by the following examples, which in no way are intended to limit the scope of the invention:

### EXAMPLES

Materials and methods

Materials—TLC silica gel 60 (aluminum) plates were from Merck (Darmstadt, Germany). All investigated glycosphingolipids were obtained in our laboratory. β-Galactosidase (Escherichia coli) was purchased from Boehringer Mannheim (Germany), Ham's F12 medium from Gibco (U.K.), ³⁵S-methionine from Amersham (U.K.) and FCS (fetal calf serum) was from Sera-Lab (England). β4-Galactosidase (Streptococcus pneunioniae), β-N-acetylhexosaminidase (Streptococcus pneumoniae) and sialidase (Arthrobacter ureafaciens) were from Oxford GlycoSystems (Abington, U.K.). The clinical isolates of Helicobacter pylori (strains 002 and 032) obtained from patients with gastritis and duodenal ulcer, respectively, were a generous gift from Dr. D. Danielsson, Örebro Medical Center, Sweden. Type strain 17875 was from Culture Collection, University of Göteborg (CCUG).

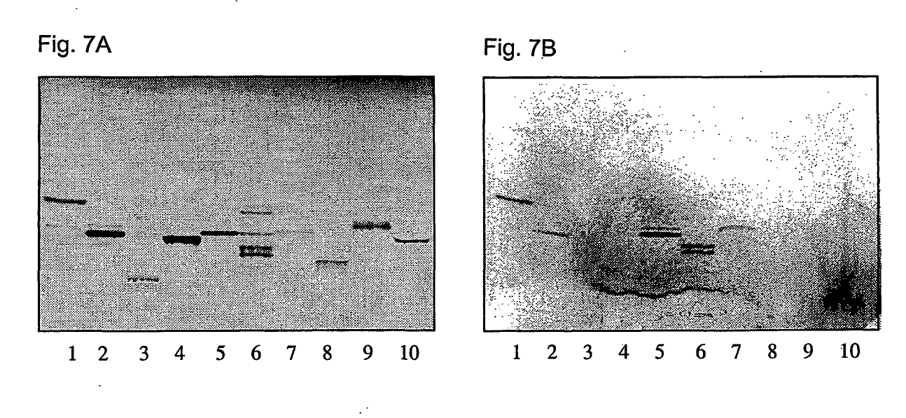

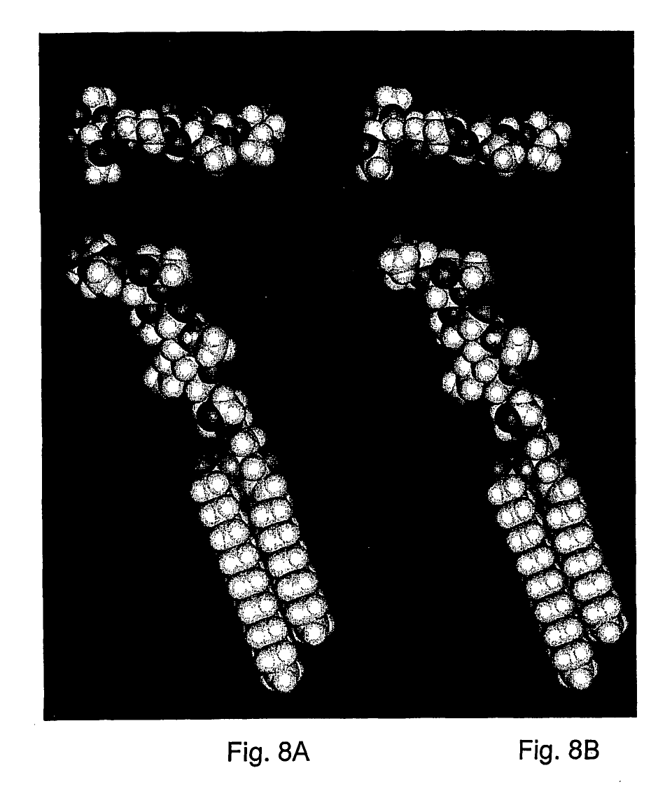

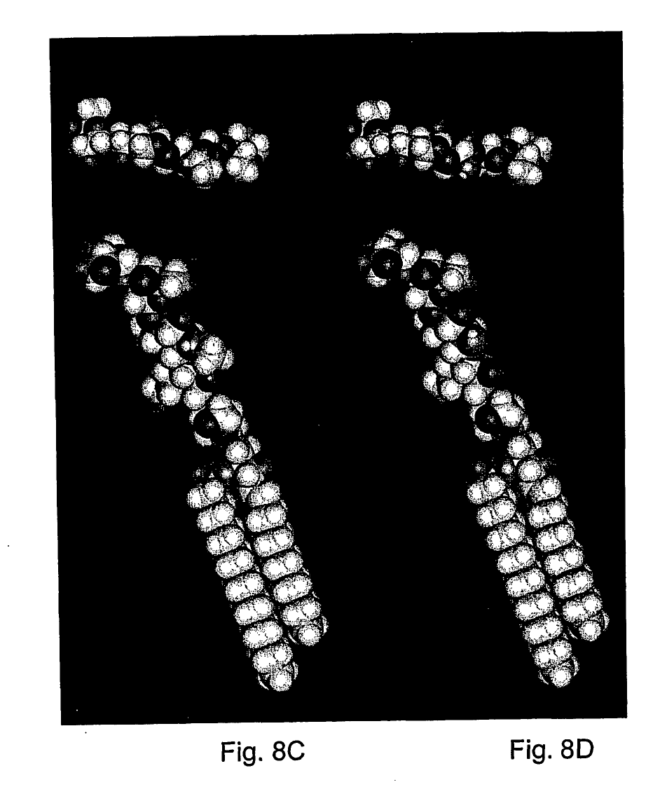

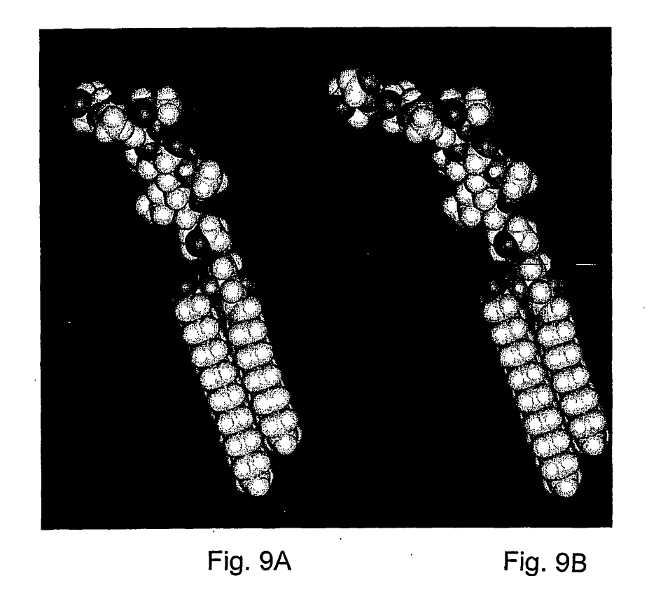

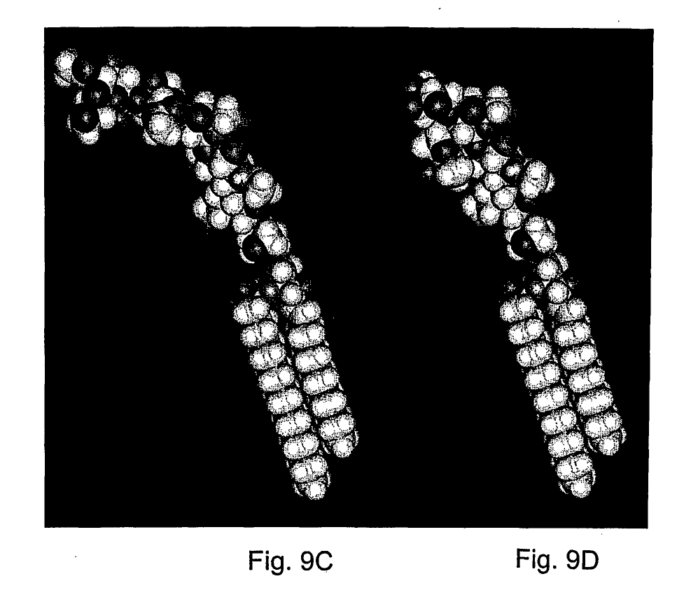

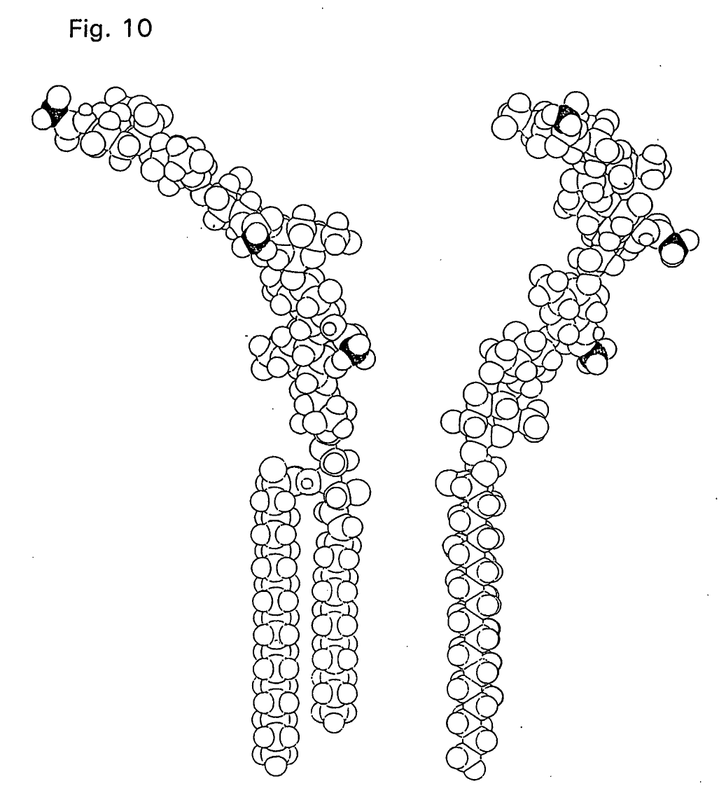

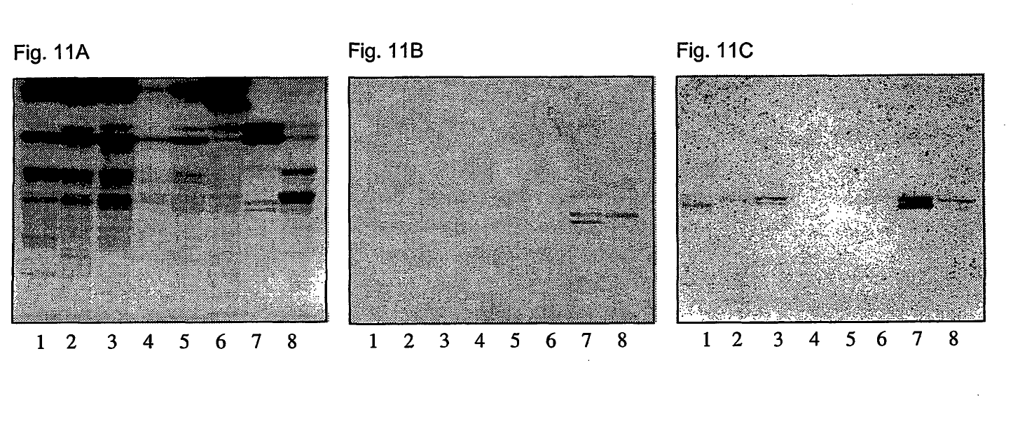

Glycosphingolipids. The pure glycosphingolipids of the experiment shown in FIGS. 7A and 7B were prepared from total acid or non-acid fractions from the sources listed in Table 2 as described in (Karlsson, 1987). In general, individual glycosphingolipids were obtained by acetylation (Handa, 1963) of the total glycosphingolipid fractions and separated by repeated silicic acid column chromatography, and subsequently characterized structurally by mass spectrometry (Samuelsson et al., 1990), NMR (Falk et al., 1979a,b,c; Koerner Jr et al., 1983) and degradative procedures (Yang and Hakomori, 1971; Stellner et al., 1973). Glycolipids derived from rabbit thymus are described below.

Purification of glycolipids. Acid glycosphingolipids were isolated from 1000 g acetone powder of rabbit thymus (Pel-Freeze Biological Inc., North Arkansas, Ark. US). The acetone powder was extracted in a Soxhlet apparatus with chroloroform/methanol 2/1 (vol/vol unless otherwise stated) for 24 h followed by chloroform/methanol/water 8/1/1 for 36 h. The extracted lipids, 240 g, were subjected to Folch separation (Folch et al., 1957) and the collected hydrophilic phase to ion-exchange gel chromatography on DE23 cellulose (DEAE, Whatman, Maidstone, UK). These isolation steps gave 2.5 g of acid glycosphingolipids. The gangliosides were separated according to number of sialic acids by ion-exchange gel with open-tubular chromatography on a glass column (50 mm i.d). The column was connected to an HPLC pump producing a concave gradient (pre-programmed gradient no 4, System Gold Chromatographic Software, Beckman Instruments Inc., Calif., USA) starting with methanol and ending with 0.5 M CH₃COONH₄ in methanol. The flow rate was 4 ml/min and 200 fractions with 8 ml in each were collected. 300-400 mg of ganglioside mixture was applied at a time to 500 g of DEAE Sepharose, (CL6, Pharmacia, Uppsala, Sweden, bed height approx. 130 mm). The monosialylated gangliosides were further separated by HPLC on a silica column, 300 mm×22 mm i.d., 120 Å pore size, 10 μm particle size (SH-044-10, Yamamura Ltd., Kyoto, Japan). Approximately 150 mg of monosialylated gangliosides were applied at time and a streight eluting gradient was used (chloroform/methanol/water from 60/35/8 to 10/103, 4 ml/min, 240 fractions).

Partial acid hydrolysis—Desialylation of gangliosides was performed in 1.5% CH₃COOH in water at 100° C. after which the material was neutralized with NaOH and dried under nitrogen. For partial degradation of the carbohydrate backbone the glycolipid was hydrolyzed in 0.5M HCl for 7 min in a boiling water bath. The material was then neutralized and partitioned in C/M/H₂O, (8:4:3, v/v)^{2.} The lower phase was collected, evaporated under nitrogen and the recovered glycolipids were used for analysis.

Preparation of pentaglycosylceramide from hexaglycosylceramide by enzyme hydrolysis—Hexaglycosylceramide (structure 2, Table 1) obtained from heptaglycosylceramide (4 mg, from rabbit thymys) (structure 1, Table 1) by acidic desialylation (see above) was redissolved in C/M (2:1) and applied to a small silica gel column (0.4×5 cm). The column was eluted with C/M/H₂O (60:35:8, v/v). Fractions of about 0.2 ml were collected and tested for the presence of carbohydrates. The recovered hexaglycosyleramide (2.0 mg) was dissolved in 1.5 ml of 0.1 M potassium phosphate buffer, pH 7.2, containing sodium taurodeoxycholate (1.5 mg/ml), MgCl₂ (0.001M) and β-galactosidase (E. coli, 500 U when assayed with 2-nitrophenyl-β-D-galactoside as a substrate), and the sample was incubated overnight at 37° C. The material was next partitioned in C/M/H₂O (10:5:3) and the glycolipid contained in the lower phase was purified using silica gel chromatography (0.4×5 cm columns) as described above for hexaglycosylceramide. To remove all contaminating detergent the chromatography was repeated twice. The final recovery of pentaglycosylceramide was 0.7 mg.

Endoglycoceramidase digestion of glycolipids (Ito and Yamagata, 1989)—The reaction mixture contained 200 μg of glycolipid, 80 μg of sodium taurodeoxycholate and 0.8 mU of enzyme in 160 μl of 50 mM acetate buffer, pH 6.0. The sample was incubated overnight at 37° C., after which water (140 μl) and C/M, (2:1, by vol., 1500 μl) were added, and the sample was shaken and centrifuged. The upper phase was dried under nitrogen, redissolved in a small volume of water and desalted on a Sephadex G-25 column (0.4×10 cm), which had been equilibrated in H₂O, and eluted with water. Fractions of about 0.1 ml were collected and tested for the presence of sugars.

Permethylation of saccharides—Permethylation was performed according to Larson et al., 1987. Sodium hydroxide was added to samples before methyl iodide as suggested by Needs and Selvendran 1993. In some experiments the saccharides were reduced with NaBH₄ before methylation. In this case the amount of methyl iodide was increased to a final proportion of DMSO (dimethylsulfoxide)/methyl iodide of 1:1 (Hansson and Karlsson, 1990).

Gas chromatography/mass spectrometry—Gas chromatography was carried out on a Hewlett-Packard 5890A Series II gas chromatograph equipped with an on-column injector and a flame ionization detector. Permethylated oligosaccharides were analyzed on a fused silica capillary column (Fluka, 11 m×0.25 mm i.d.) coated with cross-linked PS264 (film thickness 0.03 μm). The sample was dissolved in ethyl acetate and injected on-column at 80° C. The temperature was programmed from 80° C. to 390° C. at a rate of 10° C.//min with a 2 min hold at the upper temperature. Gas chromatography-mass spectrometry of the permethylated oligosaccharides was performed on a Hewlett-Packard 5890A Series II gas chromatograph interfaced to a JEOL SX-102 mass spectrometer (Hansson and Karlsson, 1990). FAB-MS analyses were performed on a JEOL SX-102 mass spectrometer. Negative FAB spectra were produced using Xe atom bombardment (10 kV) and triethanolamine as matrix.

NMR spectroscopy—Proton NMR spectra were recorded at 11.75 T on a Jeol Alpha 500 (Jeol, Tokyo, Japan) spectrometer. Samples were deuterium exchanged before analysis and spectra were then recorded at 30° C. with a digital resolution of 0.35 Hz/pt. Chemical shifts are given relative to TMS (tetramethylsilane) using the internal solvent signal.

Analytical enzymatic tests—Oxford GlycoSystems enzymatic tests were performed according to the manufacturer's recommendations except that Triton X-100 was added to each incubation mixture to final concentration of 0.3%. When a mixture of sialidase and β4-galactosidase were taken for digestion the incubation buffer from β4-galactosidase kit was used. If β-hexosaminidase was present in the digestion mixture the buffer from this enzyme kit was employed. The enzyme concentrations in the incubation mixtures were: 80 mU/ml for Hexβ4HexNAc-galactosidase (S. pneumoniae), 120 mU/ml for β-N-Acetylhexosaminidase (S. pneumoniae) and 1 U/ml for sialidase (Arthrobacter ureafaciens) The concentration of substrate was about 20 μM. Enzymatic digestion was performed overnight at 37° C. After digestion the samples were dried and desalted using small columns of Sephadex G-25 (Wells and Dittmer, 1963), 0.3 g, equilibrated in C/M/H₂O, (60:30:4.5, by vol.). Each sample was applied on the column in 2 ml of the same solvent and eluted with 2.5 ml of C/M/H₂0, (60:30:4.5) and 2.5 ml of C/M, (2:1). Application and washing solutions were collected and evaporated under nitrogen.

Other analytical methods—Hexose was determined according to Dubois et al. 1956.

De-N-acylation. Conversion of the acetamido moiety of GlcNAc/GalNAc residues into an amine was accomplished by treating various glycosphingolipids with anhydrous hydrazine as described previously (Ångström et al., 1998).

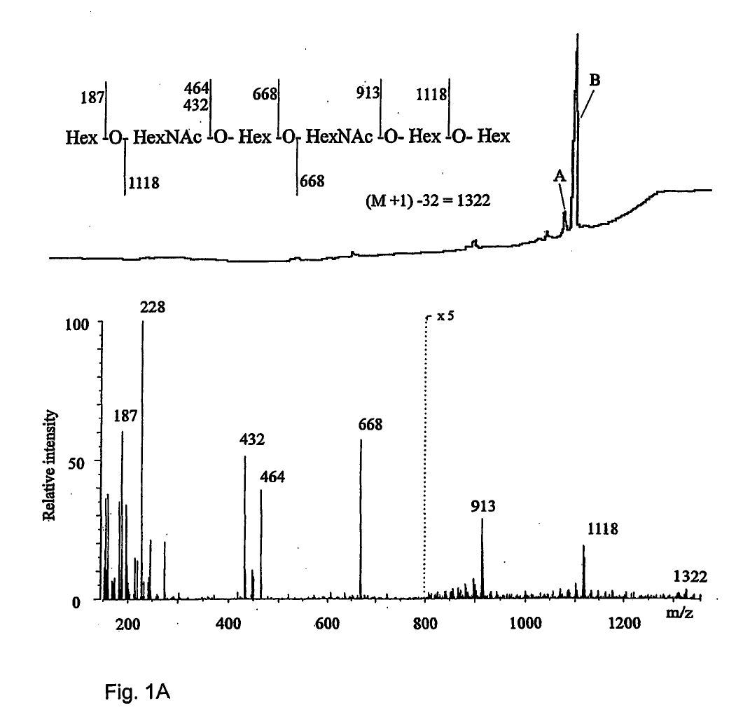

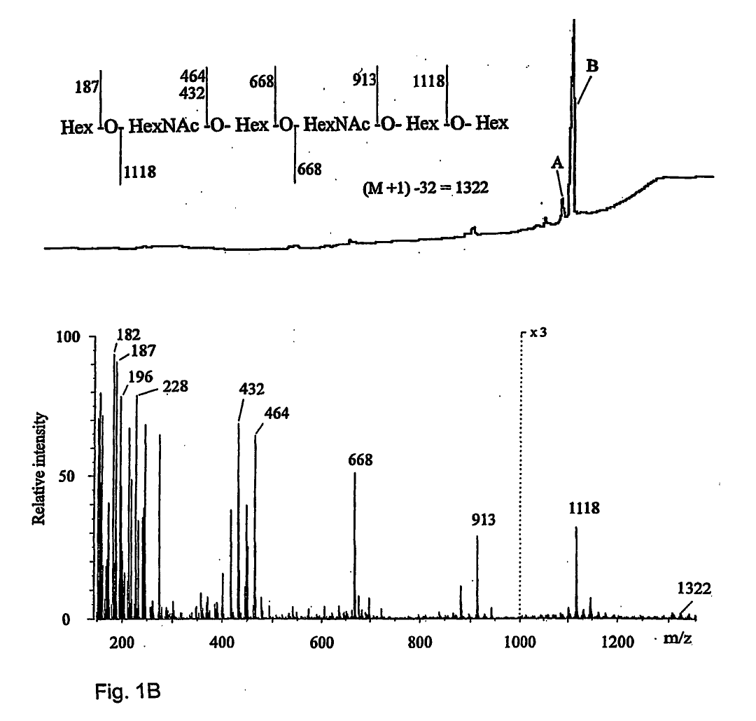

Bacterial growth. The Helicobacter pylori strains were stored at −80° C. in tryptic soy broth containing 15% glycerol (by volume). The bacteria were initially cultured on GAB-CAMP agar (Soltesz et al., 1988) under humid (98%) microaerophilic conditions (O₂: 5-7%, CO₂: 8-10% and N₂: 83-87%) at 37° C. for 48-72 h. For labeling colonies were inoculated on GAB-CAMP agar, except for the results presented in FIGS. 1A and 1B where Brucella agar (Difco, Detroit, Mich.) was used instead, and 50 μCi ³⁵S-methionine (Amersham, U.K.), diluted in 0.5 ml phosphate-buffered saline (PBS), pH 7.3, was sprinkled over the plates. After incubation for 12-24 h at 37° C. under microaerophilic conditions, the cells were scraped off, washed three times with PBS, and resuspended to 1×10⁸ CFU/ml in PBS. Alternatively, colonies were inoculated (1×10⁵ CFU/ml) in Hamns F12 (Gibco BRL, U.K.), supplemented with 10% heat-inactivated fetal calf serum (Sera-Lab). For labeling, 50 μCi ³⁵S-methionine per 10 ml medium was added, and incubated with shaking under microaerophilic conditions for 24 h. Bacterial cells were harvested by centrifugation, and purity of the cultures and a low content of coccoid forms was ensured by phase-contrast microscopy. After two washes with PBS, the cells were resuspended to 1×10⁸ CFU/ml in PBS. Both labeling procedures resulted in suspensions with specific activities of approximately 1 cpm per 100 Helicobacter pylori organisms.

TLC bacterial overlay assay. Thin-layer chromatography was performed on glass- or aluminum-backed silica gel 60 HPTLC plates (Merck, Darmstadt, Germany) using chloroform/methanol/water 60:35:8 (by volume) as solvent system. Chemical detection was accomplished by anisaldehyde staining (Waldi, 1962). The bacterial overlay assay was performed as described previously (Hansson et al., 1985). Glycosphingolipids (1-4 μg/lane, or as indicated in the figure legend) were chromatographed on aluminum-backed silica gel plates and thereafter treated with 0.3-0.5% polyisobutylmethacrylate in diethylether/n-hexane 1:3 (by volume) for 1 min, dried and subsequently soaked in P13S containing 2% bovine serum albumin and 0.1% Tween 20 for 2 h. A suspension of radio-labeled bacteria (diluted in PBS to 1×10⁸ CFU/ml and 1-5×10⁶ cpm/ml) was sprinkled over the chromatograms and incubated for 2 h followed by repeated rinsings with PBS. After drying the chromatograms were exposed to XAR-5 X-ray films (Eastman Kodak Co., Rochester, N.Y., USA) for 12-72 h.

TLC protein overlay assays. ¹²⁵I-labeling of the monoclonal antibody TH2 and the lectin from Erythrina cristagalli (Vector Laboratories, Inc., Burlingame, Calif.) was performed by the Iodogen method (Aggarwal et al., 1985), yielding an average of 2×10³ cpm/μg. The overlay procedure was the same as described above for bacteria except Tween was not used and that ¹²⁵I-labeled protein, diluted to approximately 2×10³ cpm/μl with PBS containing 2% bovine serum albumin, was used instead of a bacterial suspension.

Molecular modeling. Minimum energy conformers of the glycosphingolipids listed in Table 1 were calculated within the Biograf molecular modeling program (Molecular Simulations Inc.) using the Dreiding-II force field (Mayo et al., 1990) on a Silicon Graphics 4D/35TG workstation. Partial atomic charges were generated using the charge equilibration method (Rappé and Goddard III, 1991), and a distance dependent dielectric constant (ε=3.5 r) was used for the Coulomb interactions. In addition a special hydrogen bonding term was used in which the maximal interaction (D_{hb}) was set to −4 kcal mol⁻¹. The dihedral angles of the Glcβ1Cer linkage are defined as follows: Φ=H-1-C-1-O-1-C-1, Ψ=C-1-O-1-C-1-C-2 and θ=O-1-C-1-C-2-C-3 starting from the glucose end (see Nyholm and Pascher, 1993).

The oligosaccharide GlcNAcβ3Galβ4GlcNAc was synthesised from Galβ4GlcNAc (Sigma, St. Louis, USA) and GlcNAcβ3Galβ4GlcNAcβ6GlcNAc was synthesised from Galβ4GlcNAcβ6GlcNAc by incubating the acceptor saccharide with human serum β3-N-acetylglucosaminyltransferase and UDP-GlcNAc in presence of 8 mM MnCl₂ and 0.2 mg/ml ATP at 37 degree of Celsius for 5 days in 50 mM TRIS-HCl pH 7.5. Galβ4GlcNAcβ6GlcNAc was obtained from GlcNAcβ6GlcNAc (Sigma, St Louis, USA) by incubating the disaccharide with β4Galactosyltransferase (bovine milk, Calbiochem., Calif., USA) and UDP-Gal in presence of 20 mM MnCl₂ for several hours in 50 mM MOPS—NaOH pH 7.4. Hexasaccharide Galβ3GlcNAcβ3Galβ4GlcNAcβ3Galβ4Glc (1 mg, from Dextra labs, UK)) was treated with 400 mU β3/6-galactosidase (Calbiochem., Calif., USA) overnight as suggested by the producer. The oligosaccharides were purified chromatographically and their purity was assessed by MALDI-TOF mass spectrometry and NMR. Galα3Galβ4GlcNAcβ3Galβ4Glc was from Dextra laboratories, Reading, UK. The glyncolipid GlcAβ3Galβ4GlcNAcβ3Galβ4GlcβCer (Wako Pure Chemicals, Osaka, Japan) was reduced to Glcβ3Galβ4GlcNAcβ3Galβ4GlcβCer as described in Lanne et al 1995. The glycolipid derivative Glc(A-methylamide)β3Galβ4GlcNAcβ3Galβ4GlcβCer was produced by amidatation of the carboxylic acid group of the glucuronic acid of GlcAβ3Galβ4GlcNAcβ3Galβ4GlcβCer as described in Lanne et al 1995.

Results

The Heptaglycosylceramide NeuGcα3Galβ4GlcNAcβ3Galβ4GlcNAcβ3Galβ4GlcβCer was purified from rabbit thymus by HPLC as described above. The structure was characterized by NMR and mass spectrometry (data not shown). The heptasaccharide ganglioside was bound by most Helicobacter pylori isolates (about 60) tested in the laboratory of the inventors.

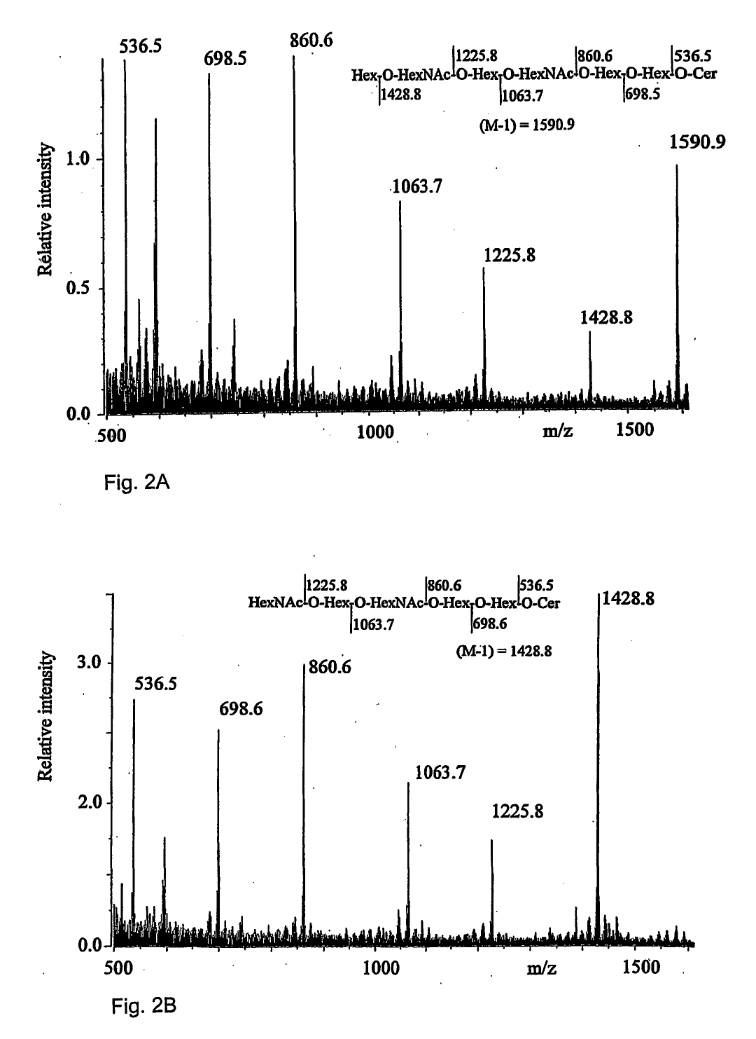

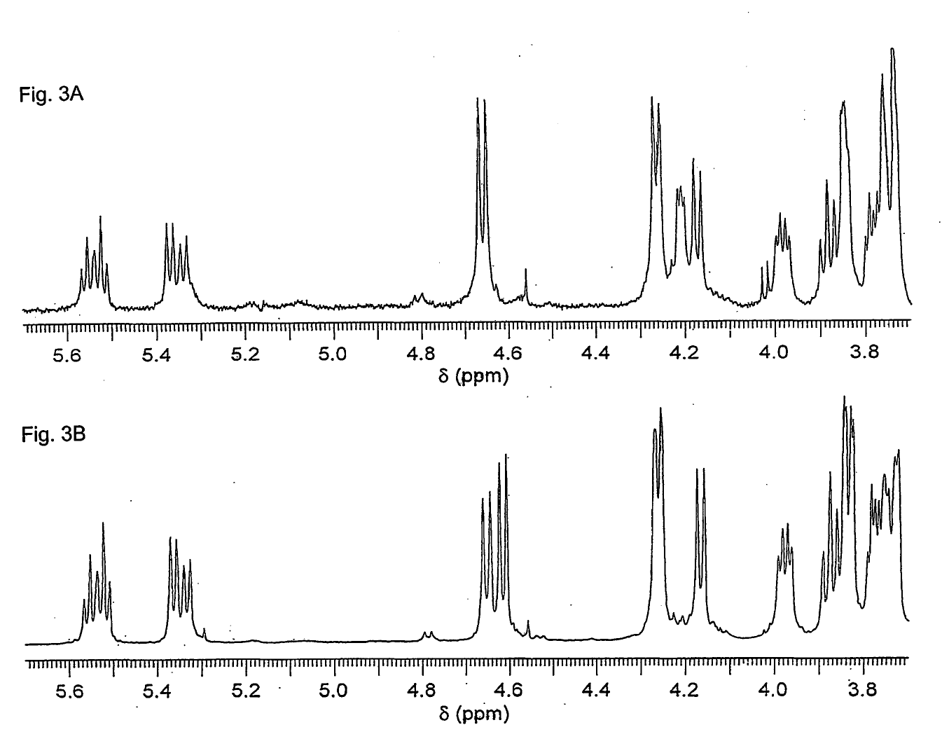

In order to detect possible minor isomeric components in the heptaglycosylceramide material, the ganglioside was desialylated, treated with endoglycoceramidase after which the released oligosaccharides were permethylated and analyzed by gas chromatography and EI/MS, (FIGS. 1A and 1B). Two saccharides were identified in the six-sugar region which showed the expected carbohydrate sequence of Hex-HexNAc-Hex-HexNAc-Hex-Hex, as confirmed by fragment ions at m/z 219, 464, 668, 913 and 1118. When the carbohydrates were converted to alditols (by reduction with NaBH₄) before methylation distinct fragment ions at m/z 235, 684 and 1133 were found in addition to the previously listed ions (data not shown). The predominant saccharide, which accounted for more than 90% of the total material (peak B, FIGS. 1A and 1B), was characterized by a strong fragment ion at m/z 182 confirming the presence of β4GlcNAc (neolacto series, type 2 carbohydrate chain). The minor saccharide (peak A, FIGS. 1A and 1B) gave a spectrum typical for type-1 chain (lacto series) with a very weak fragment ion at m/z 182 and a strong fragment ion at m/z 228. The preparation also contained traces of other sugar-positive substances which might be 4- and 5-sugar-containing saccharides of the same series. Fucose-containing saccharides were not found in the mixture. The purity of the asialoganglioside was tested also by FAB/MS and NMR spectroscopy. The negative FAB/MS of the hexaglycosylceramide (FIG. 2A) confirmed the predicted carbohydrate sequence and showed that the ceramides were composed mainly of sphingosine and C16:0 fatty acid (m/z 536.5). The NMR spectrum obtained of hexaglycosylceramide (FIG. 3A) showed four major doublets in the anomeric region with β-couplings (J˜8 Hz). They had an intensity ratio of 2:2:1:1. The signals at 4.655 ppm (GlcNAcβ3), 4.256 ppm (internal Galβ4), 4.203 ppm (terminal Galβ4) and 4.166 ppm (Glcβ) were in agreement with results previously published for nLcOse₆-Cer (Clausen et al., 1986). There was also a small doublet at 4.804 ppm, which together with a small methyl signal at 1.81 ppm (seen as a shoulder on the large type 2 methyl resonance) indicated the presence of a small fraction of type 1 chain. Due to the overlap in the 4.15 to 4.25 ppm region the position and distribution of this type 1 linkage could not be determined. The total amount of type 1 linkage was roughly 10%. As the amount of type 1 chain in the pentaglycosylceramide obtained from hexaglycosylceramide by β-galacosidase digestion also was approximately 5% (FIG. 3B) it seems likely that the type 1 linkage was evenly distributed between the internal and external parts of the saccharide chain, i.e. 5% of the glycolipids could be type1-type 1.

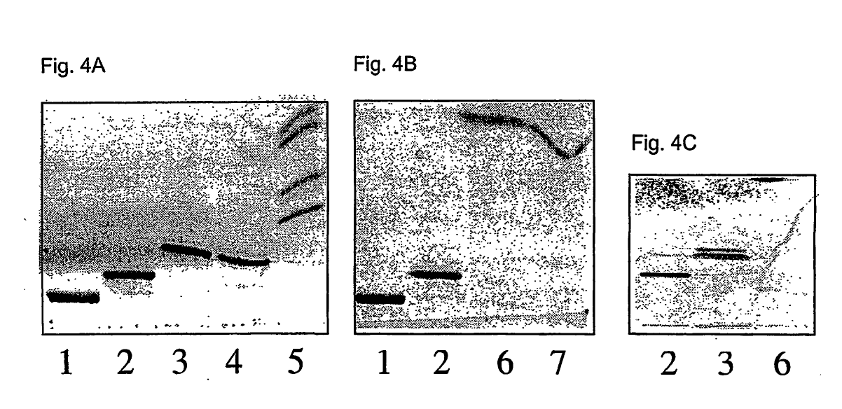

To find out if the binding activity of the glycolipid was associated with the predominant neolacto (type 2) structure the asialo-glycolipid was treated with β4-galactosidase and β-hexosaminidase, and the products were investigated by TLC and by overlay tests (FIGS. 4A, 4B and 4C). As expected, the first enzyme converted the hexaglycosylceramide to a pentaglycosylceramide (4A, lane 3) and the mixture of the two enzymes degraded the material to lactosylceramide (4B, lane 6). According to visual evaluation of the TLC plates both reactions were complete or almost complete. The same results were obtained for sialidase- and acid-treated material. The β4-galactosidase degradation of hexaglycosylceramide was accompanied by disappearance of the Helicobacter pylori binding activity in the region of this glycolipid on TLC plates with simultaneous appearance of a strong activity in the region of pentaglycosylceramides (4C, lane 3). Further enzymatic degradation of the pentaglycosylceramide resulted in the disappearance of binding activity in this region. Appearance of binding activity in the four-sugar region was not observed. The sensitivity of the chemical staining of TLC plates is too low to allow trace substances to be observed.

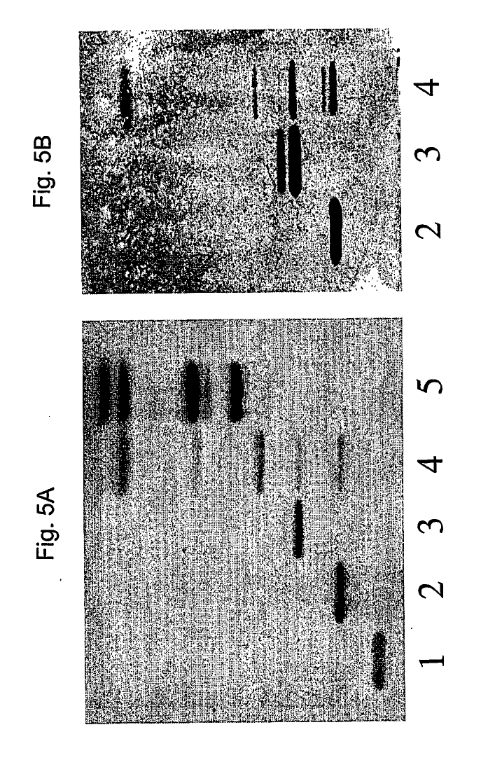

In a separate experiment the parent ganglioside was subjected to partial acid degradation and the released glycolipids were investigated for Helicobacter pylori binding activity. FIGS. 5A and 5B show TLC of the hydrolyzate (5A) and the corresponding autoradiogram (5B) after overlay of the hydrolyzate with ³⁵S-labeled Helicobacter pylori. Glycolipids located in the regions of hexa-, penta-, tetra- and diglycosylceramides displayed binding activity, whereas triglycosylceramide was inactive.

The binding of the hexa-, penta-, tetraglycosylceramides were similar when tested with at least three Helicobacter pylori strains (17875, 002 and 032).

The strongly binding pentaglycosylceramide produced after detachment of the terminal galactose from hexaglycosylceramide and purification by silica gel chromatography was investigated in greater detail. The negative ion FAB/MS spectrum of this glycolipid confirmed a carbohydrate sequence of HexNAc-Hex-HexNAc-Hex-Hex- and showed the same ceramide composition as the hexaglycosylceramide (FIG. 2B). The proton NMR spectrum obtained for the pentaglycosylceramide (FIG. 3B) had five major β-doublets in the anomeric region: at 4.653 ppm (internal GlcNAcβ3), 4.615 ppm (terminal GlcNAcβ3), 4.261 ppm (double intensity, internal Galβ4), 4.166 (Glcβ), consistent with GlcNAcβ3Galβ4GlcNAcβ3Galβ4GlcβCer and also in perfect agreement with the six sugar compound having been stripped of its terminal Galβ. There is also a small β-doublet at 4.787 ppm corresponding to 3-substituted GlcNAcβ (type 1 chain). The expected methyl signal was also seen as a shoulder on a much larger methyl signal at 1.82 ppm, but overlap prohibits quantitation of these signals. From the integral of the anomeric proton it can be calculated that 6% of the glycolipid contained type 1 chain. Thus the relative proportion of type 2 and type 1 carbohydrate chains was similar to that of the six sugar glycolipid. The two spots visible on TLC plates both in the hexa- and pentaglycosyl fractions reflected a ceramide heterogeneity rather than differences in sugar chain composition as judged by their susceptibility to β4-galactosidase. The upper penta-region spot appeared both after unselective hydrolysis of the asialoganglioside and selective splitting of 4-linked galactose from the asialoproduct. Furthermore, when hexaglycosylceramide with a high content of the upper chromatographic subfraction was degraded by β4-galactosidase and β-hexosaminidase the resulting lactosylceramide gave two distinct chromatographic bands. Chromatographically homogenous hexaglycosylceramide resulted in only one lactosylceramide band. Both upper and lower subfractions in the penta-region were highly active as shown by overlay tests.

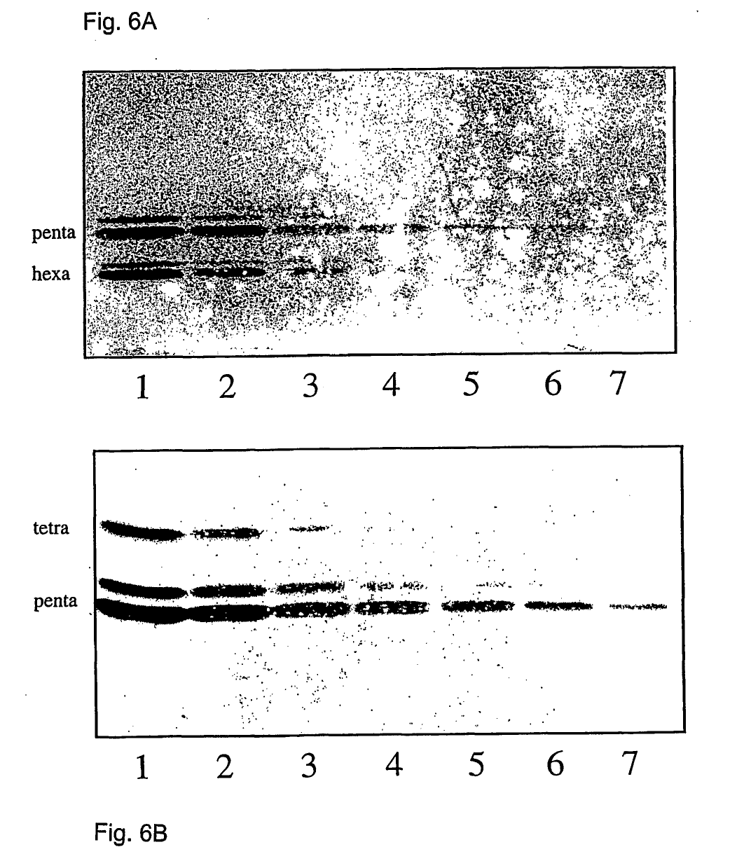

Glycosphingolipids of the neolacto series with 6, 5 and 4 sugars (structures 2, 4 and 5, Table I) were examined by semi-quantitative tests using the TLC overlay procedure. The glycolipids were applied on silica gel plates in series of dilutions and their binding to Helicobacter pylori was evaluated visually after overlay with labeled bacteria and autoradiography (FIGS. 6A and 6B). The most active species was pentaglycosylceramide, which gave a positive response on TLC plates in amounts down to 0.039 nmol/spot (mean value calculated from 7 experiments, standard deviation δₙ₋₁=0.016 nmol). Hexa- and tetraglycosylceramides bound Helicobacter pylori in amounts of c:a 0.2 and 0.3 nmoles of glycolipid/spot, respectively.

The binding of Helicobacter pylori to higher glycolipids of the investigated series was highly reproducible. The binding frequency for Helicobacter pylori, strain 032, recorded for pentaglycosyl- and hexaglycosylceramides was ˜90% (total number of plates was about 100).

Binding Assays Revealing the Isoreceptors and Specificity of the Binding (FIGS. 7A and 7B.)

In addition to the seven-sugar glycosphingolipid from rabbit thymus having a neolacto core, NeuGcα3Galβ4GlcNAcβ3Galβ4GlcNAcβ3Galβ4GlcβCer, and tetra- to hexaglycosylceramides derived thereof, the binding specificity could involve other glycolipids from the neolacto series.

The binding of Helicobacter pylori (strain 032) to purified glycosphingolipids separated on thin-layer plates using the overlay assay is shown in FIGS. 7A and 7B. These results together with those from an additional number of purified glycosphingolipids are summarized in Table 2. The binding of Helicobacter pylori to neolactotetraosylceramide (lane 1) and the five- and six-sugar glycosphingolipids (lanes 5 and 6) derived from NeuGcα3Galβ4GlcNAcβ3Galβ4GlcNAcβ3Galβ4GlcβCer is identical to results above. Unexpectedly, however, binding was also found for GalNAcβ3Galβ4GlcNAcβ3Galβ4GlcβCer (x₂ glycosphingolipid, lane 7) and the de-fucosylated A6-2 glycosphingolipid GalNAcα3 Galβ4GlcNAcβ3Galβ4GlcβCer (no. 12, Table 2). Together with the finding that Galα3Galβ4GlcNAcβ3Galβ4GlcβCer (B5 glycosphingolipid, lane 2) also is binding-active, these results suggest the possibility of cross-binding rather than the presence of multiple adhesins specific for each of these glycosphingolipids (see below). Furthermore, the only extension of the different five-sugar-containing glycosphingolipids just mentioned that was tolerated by the bacterial adhesin was Galβ4 to the thymus-derived GlcNAcβ3-terminated compound (lane 6). Other elongated structures, as the NeuAc-x₂ (lane 8) and GalNAcβ3-B5 (no. 25, Table 2), were thus all found to be non-binding. It may be further noticed that the acetamido group of the internal GlcNAcβ3 in B5 is essential for binding since de-N-acylation of this moiety by treatment with anhydrous hydrazine leads to complete loss of binding (lane 3) as is the case also when neolactotetraosylceramide is similarly treated (no. 6, Table 2).

Cross-binding of five-sugar glycosphingolipids. In order to understand the binding characteristics of the different neolacto-based glycosphingolipid molecules used in this study the conformational preferences of active as well as inactive structures were investigated by molecular modeling. FIGS. 8A, 8B, 8C and 8D show the x₂ glycosphingolipid together with three other sequences: defucosylated A6-2, B5 and de-N-acylated B5, which, except for the chemically modified B5 structure, show similar binding strengths. Also the five-sugar glycosphingolipid from rabbit thymus (see FIG. 9A) should be included in this comparison since this structure differs only at position four of the terminal residue compared with the x₂ structure and is equally active. The four active structures all have neolacto cores which thus are terminated by GalNAcβ3, GalNAcα3, Galα3 and GlcNAcβ3, respectively. The minimum energy conformers of these structures were generated as described previously (Teneberg et al., 1996). Other minimum energy structures given in Table 2 are based on earlier results found in the literature (Bock et al., 1985; Meyer, 1990; Nyholm et al., 1989). Regarding sialic acid-terminated glycosphingolipids the synclinal conformation was adopted for the glycosidic dihedral angles of α3-linked residues as seen in, e.g., FIG. 9C, but the effect of other conformations (Siebert et al., 1992), in particular the anticlinal one, was also tested. Also for the α6-linked variant several low energy conformers (Breg et al., 1989) were generated for the same purpose.

As mentioned above, the fact that there are four binding-active five-sugar glycosphingolipids (nos. 10-13, Table 2), all having a neolacto core, suggests that cross-binding to the same adhesin site may be the reason behind these observations. At first glance, however, it might seem surprising that the B5 glycosphingolipid, which differs at the terminal position in comparison with the five-sugar compound obtained from rabbit thymus, the former having a Galα3 and the latter a GlcNAcβ3, is equally active and should be included within the binding specificity of the neolacto series. Despite the fact that these two terminal saccharides differ also in their anomeric linkage it is seen (FIGS. 8C and 9A) that the minimum energy structures topographically are very similar, the differences being that Galα3 lacks an acetamido group, has the 4-OH in the axial position and its ring plane raised slightly above the corresponding plane in the five-sugar compound. However, neither the 4-OH position nor the absence/presence of an acetamido group appear to be crucial for binding to occur, since also the x₂ and defucosylated A6-2 glycosphingolipids (FIGS. 8A, B), which are terminated by GalNAcβ3 and GalNAcα3, respectively, have similar affinities for the Helicobacter pylori adhesin. In the light of these findings also Galβ3Galβ4GlcNAcβ3Galβ4GlcβCer, which has been isolated from human erythrocytes (Stellner and Hakomori, 1974), would be expected to bind the bacterial adhesin. In the light of the rules of binding also three other terminal monosaccharides in Helicobacter pylori binding epitopes are possible trisaccharide binding epitopes, namely GlcNAcα3Galβ4GlcNAc, Glcβ3Galβ4GlcNAc and Glcα3Galβ4GlcNAc. Such compounds are not known from human tissues so far, but could rather represent analogues of the natural receptor. Neither the Galβ3Galβ4GlcNAc-glycolipid nor the three analogs were unfortunately available for testing.

The neolacto seven-sugar compound,NeuGcα3Galβ4GlcNAcβ3Galβ4GlcNAcβ3Galβ4GlcβCer, was also subjected to molecular modeling. FIG. 10 shows two different projections of the minimum energy structure with the GlcβCer linkage in an extended conformation. The sialic acid was given the syn clinal conformation but the anti conformer is also likely in unbranched structures (Siebert et al., 1992). The sialic acid appears to have little influence on the binding activity towards Helicobacter pylori as compared with the six-sugar compound, 9B. Comparison of the first projection with FIGS. 9A and 9B suggests that the same binding epitope is also available in the seven-sugar structure.

Delineation of the neolacto binding epitope. The relative binding strength of the structures obtained by chemical and enzymatic degradation of the rabbit thymus seven-sugar compound (nos. 1, 5, 10, and 21, Table 2) suggest that the three-sugar sequence GlcNAcβ3Galβ4GlcNAcβ3 may constitute the minimal binding sequence. Thus, in the six-sugar compound an inhibitory effect from the terminal Galβ4 is expected, whereas for neolactotetraosylceramide lack of a terminal GlcNAcβ3 reduces the binding strength since only two out of three sugars in the epitope are present. The essentiality of the internal GlcNAcβ3 is clearly shown by the loss of bacterial binding both to neolactotetraosylceramide and B5 following de-N-acylation of the acetamido group to an amine (nos. 6 and 14, Table 2). This non-binding may occur either by loss of a favorable interaction between the adhesin and the acetamido moiety and/or altered conformational preferences of these glycosphingolipids. However, it is difficult to envision a situation where an altered orientation of the internal Galβ4 would sterically hinder access to the binding epitope. Thus, having established that the minimal binding sequence must encompass the GlcNAcβ3Galβ4GlcNAcβ3 sequence it is now easy to rationalize the absence of binding for P₁, H5-2 and the two sialylparagloboside structures (nos. 15, 18-20, Table 2) since these extensions interfere directly with the proposed binding epitope. Also the glycosphingolipid from bovine buttermilk (Teneberg et al., 1994), which has a β6-linked branch of Galβ4GlcNAcβ attached to the internal Galβ4 of neolactotetraosylceramide (no. 26, Table 2), is non-binding due to blocked access to the binding epitope.

Elongation of the different binding-active five-sugar sequences in Table 2 shows that only addition of Galβ4 to the thymus-derived structure is tolerated, in accordance with the observation that the 4-OH position may be either equatorial or axial, but with an ensuing loss of binding affinity due to steric interference. Addition of either NeuAcα3 to x₂ or GalNAcβ3 to B5 thus results in complete loss of binding (nos. 24 and 25, Table 2). It is further seen that the negative influence of a Fucα2 unit as in H5-2 is confirmed by the non-binding of Helicobacter pylori both to A6-2 and B6-2 (nos. 22 and 23, Table 2). Concerning the elongated structure (no. 28, Table 2), terminated by the same trisaccharide found in B5, it must, as in B5, be this terminal trisaccharide that is responsible for the observed binding although a second internal binding epitope also is present. However, binding to the internal epitope can most likely be excluded since the penultimate Galβ4 would be expected to is obtained or not depends, however, both on the type of strain and growth conditions (Miller-Podraza et al., 1996, 1997a, b).

To summarize, the binding epitope of the neolacto series of glycosphingolipids has to involve the three-sugar sequence GlcNAcβ3Galβ4GlcNAcβ3 in order to obtain maximal activity. From a comparison of the binding pattern of the potential isoreceptors used in this study it can be deduced from the structures shown in FIGS. 8A-D and 9A-D that nearly all of this trisaccharide is important for binding to occur, excepting the acetamido group of the terminal GlcNAcβ3 and the 4-OH on the same residue, which are non-crucial.

Biological presence of the receptors. Of the four five-sugar glycosphingolipids that in vitro may function interchangeably as receptors for Helicobacter pylori only x₂ occurs naturally in human tissue but has as yet not been found to be present in the gastric mucosa, excepting a case of gastric cancer where it was identified in the tumor tissue (Kannagi et al., 1982b). A study by Thorn et al., 1992, showed, however, that the x₂ glycosphingolipid and elongated structures having a terminal GalNAcβ3Galβ4GlcNAcβ sequence are present in several human tissues, but gastric epithelial tissue was unfortunately not among the ones investigated. Thin-layer chromatogram overlay with the GalNAcβ3Galβ4GlcNAcβ-specific monoclonal antibody TH2 of preparations of total non-acid glycosphingolipids from epithelial cells of human gastric mucosa of several blood group A individuals (lanes 1-6) was therefore performed (FIG. 11B). No detectable binding, however, was observed to the glycosphingolipids derived from stomach epithelium using this assay. The corresponding overlay using the Galβ4GlcNAc-binding lectin from E. cristagalli is shown in FIGS. 11A, 11B and 11C. Of the different glycosphingolipid preparations of gastric epithelial origin the first three lanes show weak binding to bands in the four-sugar region, which probably correspond neolactotetraosylceramide, but no detectable binding of Helicobacter pylori to these bands was discerned due to the low amounts of this glycosphingolipid (Teneberg et al., 2001).

Furthermore, the sequence Galα3Galβ4GlcNAcβ, whether present in B5 glycosphingolipid or in the elongated structure discussed above (no. 28, Table 2), is possibly not found in normal human tissue due to non-expression of the transferase responsible for the addition of Galα3 (Larsen et al., 1990). One is therefore left with the conclusion that if target receptor(s), carrying the binding epitope identified above, are present on the surface of the gastric epithelial cells they may be based on repetitive N-acetyllactosamine elements in glycoproteins and not on lipid-based structures.

However, it is known that Helicobacter pylori strains associated with peptic ulcer, as the strain mainly used here, stimulates an inflammatory response from granulocytes, even when the bacteria are nonopsonized (Rautelin et al., 1994a, b). The initial event in the phagocytosis of the bacterium most likely involves specific lectin-like interactions resulting the agglutination of the granulocytes (Ofek and Sharon, 1988). Subsequent to the phagocytotic event oxidative burst reactions occur which may be of consequence for the pathogenesis of Helicobacter pylori-associated diseases (Babior, 1978). Several acid and non-acid glycosphingolipids from granulocytes, having both a neolacto core and repeating lactosamine units, including no. 21. in Table 2 and the sialylated seven-sugar compound (no. 27, Table 2), where the acetamido group of the sialic acid is in the acetyl form, have been isolated and characterized (Fukuda et al., 1985; Stroud et al., 1996) and may thus act potential receptors for Helicobacter pylori on the white blood cell surface. Furthermore, also the x₂ glycosphingolipid has been isolated from the same source (Teneberg, S., unpublished).

Returning to FIG. 11B it is seen that the monoclonal antibody TH2 indeed binds to bands in the five-sugar region, both for granulocytes and erythrocytes (lanes 7 and 8, respectively), which may correspond to the x₂ glycosphingolipid (Teneberg, S., unpublished; Thorn et al., 1992; Teneberg et al., 1996). Similarly, neolactotetraosylceramide is found to be present both in granulocytes and erythrocytes when using the E. cristagalli lectin instead in the overlay assay (FIG. 11C, lanes 7 and 8). In these two cases Helicobacter pylori binds to neolactotetraosylceramide (Bergstöm, J., unpublished). For granulocytes a further rather weak band in the six-sugar region, probably corresponding to neolactotetraosylceramide extended by one N-acetyllactosamine unit (cf. no. 21, Table 2), is found in accordance with the results of Fukuda et al., 1985. Whether these glycosphingolipids are prime targets in the agglutination process referred to above remains, however, to be elucidated.

Analysis of Neoglycolipids and Novel Glycolipids

The oligosaccharides GlcNAcβ3Galβ4GlcNAc, GlcNAcβ3Galβ4GlcNAcβ6GlcNAc, Galα3Galβ4GlcNAcβ3Galβ4Glc and GlcNAcβ3Galβ4GlcNAcβ3Galβ4Glc and maltoheptaose (Sigma, Saint Louis, USA) were reductively aminated with 4-hexadecylaniline (abbreviation HDA, from Aldrich, Stockholm, Sweden) by cyanoborohydride (Halina Miller-Podraza, to be published later). The products were characterized by mass spectrometry and were confirmed to be GlcNAcβ3Galβ4GlcNAc(red)-HDA, GlcNAcβ3Galβ4GlcNAcβ6GlcNAc(red)-HDA, Galα3Galβ4GlcNAcβ3Galβ4Glc(red)-HDA, GlcNAcβ3Galβ4GlcNAcβ3Galβ4Glc(red)-HDA and maltoheptaose(red)-HDA [where “(red)-” means the amine linkage structure formed by reductive amination from the reducing end glucoses of the saccharides and amine group of the hexadecylaniline (HDA)]. The compounds Galα3Galβ4GlcNAcβ3Galβ4Glc(red)-HDA and GlcNAcβ3Galβ4GlcNAcβ3Galβ4Glc(red)-HDA had clear binding activity and GlcNAcβ3Galβ4GlcNAcβ6GlcNAc(red)-HDA had strong binding activity with regard to Helicobacter pylori in TLC overlay assay described above, while the GlcNAcβ3Galβ4GlcNAc(red)-HDA and maltoheptaose(red)-HDA were weakly binding or inactive. The example shows that the tetrasaccharide GlcNAcβ3Galβ4GlcNAcβ3Gal is a structure binding to Helicobacter pylori. The reducing end Glc-residue is probably not needed for the binding because the reduction destroys the pyranose ring structure of the Glc-residue. In contrast, the intact ring structure of reducing end GlcNAc is needed for good binding of the trisacharide GlcNAcβ3Galβ4GlcNAc.

The a biosynthetic precursor analog of NHK-1 glycolipid GlcAβ3Galβ4GlcNAcβ3Galβ4GlcβCer, and novel glycolipids Glcβ3Galβ4GlcNAcβ3Galβ4GlcβCer and Glc(A-methylamide)β3Galβ4GlcNAcβ3Galβ4GlcβCer were tested in TLC overlay assay and were observed to be binding active with regard to Helicobacter pylori. Glc(A-methylamide) means glucuronic acid derivative wherein the carboxylic acid group is amidated with methylamine. The Glcβ3Galβ4GlcNAcβ3Galβ4GlcβCer structure had strong binding towards H. pylori and Glc(A-methylamide)β3Galβ4GlcNAcβ3Galβ4GlcβCer had very strong binding to Helicobacter pylori.

Production of GlcAβ3Galβ4Glc(NAc) by transglycosylation The acceptor saccharide Galβ4Glc or Galβ4GlcNAc (about 10-20 mM) is incubated with 10 fold molar excess paranitrophenyl-beta-glucuronic acid and bovine liver β-glucuronidase (20 000 U, Sigma) in buffer having pH of about 5 for two days at 37 degrees of Celsius stirring the solution. The product is purified by HPLC.

References

Aggarwal, B. B., Eessalu, T. E. and Hass, P. E. (1985) Nature, 318, 665-667.

Andersson, B., Porras, O., Halson, L.Å., Lagergård, T., and Svanborg-Edén, C. (1986) J. Inf. Dis. 153, 232-7

Ångstrom, J., Teneberg, S., Abul Milh, M., Larsson, T., Leonardsson, I., Olsson, B. -M., Ölwegård Halvarsson, M., Danielsson, D., Näslund, I., Ljung, Å., Wadström, T. and Karlsson, K. -A. (1998) Glycobiology, 8, 297-309.

Ascencio, F., Fransson, L -Å. and Wadström, T. (1993) J. Med. Microbiol., 38, 240-244.

Appelmelk, B. J., Faller, G., Clayes, D., Kirchner, T., and Vandenbroucke-Grauls, C. M. J. E. (1998) Immol. Today 19, 296-299.

Avenaud, P., Marais, A., Monteiro, L., Le Bail, B., Biolac Saga, P., Balabaud, C., and Mégraud, F. (2000) Cancer 89, 1431-1439.

Axon, A. T. R. (1993) J. of Antimicrobial Chemotherapy, 32, 61-68.

Babior, B. M. (1978) N. Eng. J Med., 298, 659-668.

Blaser, M. J. (1992) Eur. J. Gastroenterol. Hepatol., 4 (suppl 1), 17-19.

Bock. K., Breimer, M. E., Brignole, A., Hansson, G. C., Karlsson, K. -A., Larson, G., Leffler, H., Samuelsson, B. E., Strömberg, N., Svanborg-Edén, C. and Thurin, J. (1985) J. Biol. Chem., 260, 8545-8551.

Borén, T., Falk, P., Roth, K. A., Larson, G. and Norrnark, S. (1993) Science, 262, 1892-1895.

Breg, J., Kroon-Batenburg, L. M. J., Strecker, G., Montreuil, J. and Viegenthart, F. G. (1989) Eur. J. Biochem., 178, 727-739.

Castagliuolo, I., La Mont, J. T., Qiu, B., Nikulasson, S. T., and Pothoulakis, C. (1996) Gastroenterology 111, 433-438.

Castronovo, V., Colin, C., Parent, B., Foidart, J. -M., Lambotte, R., and Mahieu, P. (1989) J. Natl. Cancer Inst., 81, 212-216

Charlwood, J., Tolson, D., Dwek, M., and Camillen, P. (1999) Anal. Biochem., 273, 261-77.

Clausen, H., Levery, S. B., Kannagi, R. and Hakomori, S. -i. (1986) J. Biol. Chem., 261, 1380-1387.

Chmiela, M., Wadström, T., Folkesson, H., Planeta Malecka, I., Czkwianianc, E., Recheinski, T., and Rudnicka, W. (1998) Immunol. Lett. 61, 119-125.

Claeys, D., Faller, G., Appelmelk, B. J., Negrini, R, and Kirchner, T. (1998) Gastroenterology 115, 340-347.

Correa, T. L., Fox, J., Fontham, E., Ruiz, b., Lin, Y., zaula, D., Taylor, N., Mackinley, D., deLima, E., Portilla, H., Zarama, G. (1990) Cancer 66, 596-574.

DeCross, A. J. and Marshall, B. J. (1993) Am. J. Med. Sci., 306, 381-392.

Dooley, C. P. (1993) Curr. Opin. Gastroenterol., 9, 112-117.

Dubois, M., Gilles, K. A., Hamilton, J. K., Rebers, P. A. and Smith, F. (1956) Analytical Chemistry 28, 350-356.

Dunn, B. E., Cohen, H. and Blaser, M. J. (1997) Clin. Microbiol. Rev., 10, 720-741.

Ernst, B., Hart, G. W., and Sinaÿ, P. (eds.) (2000) Carbohydrates in Chemistry and Biology, ISBN 3-527-29511-9, Wiley-VCH, Weinheim.

Eto, T., Ichikawa, Y., Nishimura, K., Ando, S. and Yamakawa, T. (1968) J. Biochem. (Tokyo), 64, 205-213.

Evans, D. G., Evans Jr, D. J., Molds, J. J., and Graham, D. Y. (1988) Infect. Immun., 56, 2896-06

Falk, K. -E., Karlsson, K. -A. and Samuelsson, B. E. (1979a) Arch. Biochem. Biophys., 192, 164-176.

Falk, K. -E., Karlsson, K. -A. and Samuelsson, B. E. (1979b) Arch. Biochem. Biophys., 192, 177-190.

Falk, K. -E., Karlsson, K. -A. and Samuelsson, B. E. (1979c) Arch. Biochem. Biophys., 192, 191-202.

Farsak, B., Yildirir, A., Akyön, Y., Pinar, A., Öc, M., Böke, E., Kes, S., and Tokgözoglu, L. (2000) J. clin. Microbiol. 38, 4408-4411.

Folch, J., Lees, M., And Sloane-Stanley, G. H. (1957) J. Biol. Chem. 226, 497-509.

Fukuda, M. N., Dell, A., Oates, J. E., Wu, P., Klock, J. C. and Fukuda, M. (1985) J. Biol. Chem., 260, 1067-1082.

Handa, S. (1963) Jap. J. Exp. Med., 33, 347-360.

Hansson, G. C., Karlsson, K. -A., Larson, G., Strömberg, N. and Thurin, J. (1985) Anal. Biochem., 146, 158-163.

Hansson, G. C. and Karlsson, H. (1990) Methods Enzymol., 193, 733-738.

Hu, J., Stults, C. L., Holmes, E. H., and Macher, B. A. (1994) Glycobiology 4, 251-7.

Ilver, D., Arnqvist, A., Ogren, J., Frick, I. M., Kersulyte, D., Incecik, E. T., Berg, D. E., Covacci, A., Engstrand, L., and Boren T. (1998) Science, 279(5349), 373-377.

Ito, M. and Yamagata, T. (1989) Methods Enzymol., 179, 488-496.

Jassel, S. V., Ardill, J. E. S., Fillmore, D., Bamford, K. B., O'Connor, F. A., and Buchanan, K. D. Q. J. Med. 92, 373-377.

Kannagi, R., Levine, P., Watanabe, K. and Hakomori, S. -i. (1982b) Cancer Res., 42, 5249-5254.

Karlsson, N. G., Olson, F. J., Jovall, P -Å, Andersch, Y., Enerback, L., and Hansson G. C. (2000) Biochem. J., 350, 805-814.

Karlsson, K. -A. (1987) Meth. Enzymol., 138,212-220.

Karlsson, K. -A. (1989) Annu. Rev. Biochem., 58, 309-350.

Karlsson, K. -A. and Larsson, G. (1981a) J. Biol. Chem. 256, 3512-3524.

Karlsson, K. -A. and Larsson, G. (1981b) FEBS Lett., 128, 71-74.

Kerr, J. R., Al-Khattaf, A., Barson, A. J., and Burnie, J. P. (2000) Arch. Child. Dis., 83, 429-434

Koerner Jr, T. A. W., Prestegard, J. H., Demou, P. C. and Yu, R. K. (1983) Biochemistry, 22, 2676-2687.

Koscielak, J., Piasek, A., Gorniak, H., Gardas, A. and Gregor, A. (1973) Eur. J. Biochem., 37, 214-215.

Laine, R. A., Stellner, K. and Hakomori, S. -i. (1974) Meth. Membr. Biol., 2, 205-244.

Lanne, B., Uggla, L., Stenhagen, G., And Karlsson, K. -A. (1995) Biochemistry 34, 1845-1850

Lanne, B., Miller-Podraza, H., Abul Milh, M., Teneberg, S., Uggla, L., Larsson, T., Leonardsson, I., Jovall, P. -Å., Bergström, J. and Karlsson, K. -A. (2001) manuscript in preparation

Larsen, R. D., Rivera-Marrero, C. A., Ernst, L. K., Cummings, R. D. and Lowe, J. B. (1990) J. Biol. Chem., 265, 7055-7061.

Larson, G., Karlsson, H., Hansson, G. C. and Pimlott, W. (1987) Carbohydr. Res., 161, 281-290

Ledeen, R. and Yu, R. K. (1978) Res. Methods Neurochem., 4, 371-410.

Lin, J. -T., Wang, J. -T., Wang, M. -S., Wu, M. -S. and Chen, C. -J. (1993) Hepato-Gastroenterol., 40, 596-599.

Lingwood, C. A., Huesca, M. and Kuksis, A. (1992) Infect. Immun., 60, 2470-2474.

Mayo, S. L., Olafsen, B. D. and Goddard III, W. A. (1990) J. Chem. Phys., 94, 8897-8909.

McKibbin, J. M., Spencer, W. A., Smith, E. L., Mansson, J. B., Karlsson, K -A, Samuelsson, B. E., Li, Y -T and Li, S. C. (1982) J. Biol. Chem., 257, 755-760.

Meyer, B. (1990) Topics Curr. Chem., 154, 141-208.

Miller-Podraza, H., Abul Milh, M., Bergström, J. and Karlsson, K. -A. (1996) Glycoconj. J., 13, 453-460.

Miller-Podraza, H., Bergström, J., Abul Milh, M. and Karlsson, K. -A. (1997a) Glycoconj. J., 14, 467-471.

Miller-Podraza, H., Abul Milh, M., Teneberg, S. and Karlsson, K. -A. (1997b) Infect. Immun., 65, 2480-2482.

Miller-Podraza, H., Abul Milh, M., Ångström, J., Jovall. P. -Å., Wilhelnsson, U., Lanne, B., Karlsson, H., and Karlsson, K. -A. (2001) manuscript in preparation.

Muzzarelli, R. A. A., Mattioli-Belmonte, M., Miliani, M., Muzzarelli, C., Gabbanelli, F., and Biagini, G. (2002) Carbohyrate Polym. 48, 15-21

Muzzarelli, R. A. A., Muzzarelli, C., Cosani, A., and Terbojevich, M. (1999) Carbohyrate Polym. 39, 361-367

Mysore, J. V., Wiggington, T., Simon, P. M., Zopf, D., Heman-Ackah, L. M. and Dubois, A. (1999) Gastroenterology, 117, 1316-1325

Naiki, M., Fong, J., Ledeen, R. and Marcus, D. M. (1975) Biochemistry, 14,4831-4836.

Nakhla, T., Fu, D., Zopf, D., Brodsky, N., and Hurt, H. (1999) British J. Nutr. 82, 361-367.

Needs, P. W. and Selvendran, R. R. (1993) Carbohydr. Res., 245, 1-10.

Nilsson, H. -O., Taneera, J., Castedal, M., Glatz, E., Olsson, R., and Wadström, T. (2000) J. Clin. Microbiol. 38, 1072-1076.

Nilsson, O., Månsson, J. -E., Tibblin, E. and Svennerholm, L. (1981) FEBS Lett., 133, 197-200.

Nomenclature of glycoproteins (1988) J. Biol. Chem., 262, 13-18.

Nomura, A. and Stemmermann, G. N. (1993) J. Gastroenterol. Hepatol., 8, 294-303.

Nyholm, P. G., Samuelsson, B. E., Breimer, M. and Pascher, I. (1989) J. Mol. Recog., 2, 103-113.

Nyholm, P. -G. and Pascher, I. (1993) Biochemistry, 32, 1225-1234.

Ofek, I. and Sharon, N. (1988) Infect. Immun., 56, 539-547.

Pakodi, F., Abdel-Salam, O. M. E., Debraceni, A., and Mozsik, G. (2000) J. Physiol. (Paris), 94, 139-152.

Parsonnet, J., Friedman, G. D., Vandersteen, D. P., Chang, Y., Vogelman, J. H., Orentreich, N. and Sibley, R. K. (1991) N. Engl. J. Med., 325, 1127-31

Rappé, A. K. and Goddard III, W. A. (1991) J. Chem. Phys., 95, 3358-3363.

Rautelin, H., Blomberg, B., Järnerot, G. and Danielsson, D. (1994a) Scand. J. Gastroenterol., 29, 128-132.

Rautelin, H., von Bonsdorff, C. -H., Blomberg, B. and Danielsson, D. (1994b) J. Clin. Pathol., 47, 667-669.

Rebora, R., Drago, F., and Parodi, A. (1995) Dermatology 191, 6-8.

Saitoh, T., Natomi, H., Zhao, W., Okuzumi, K., Sugano, K., Iwamori, M. and Nagai, Y. (1991) FEBS Lett., 282, 385-387.

Samuelsson, B. E., Pimlott, W. and Karlsson, K. -A. (1990) Meth. Enzymol., 193, 623-646.

Sears, P., and Wong, C -H. (1996) Proc. Natl. Acad. Sci., 93, 12086-12093.

Siebert, H. -C., Reuter, G., Schauer, R., von der Lieth, C. -W. and Dabrowski, J. (1992) Biochemistry, 31, 6962-6971.

Simon, P. M., Goode, P. L., Mobasseri, A., and Zopf, D. (1997) Infect. Immun. 65, 750-757

Soltesz, V., Schalen, C. and Mårdh, P. A. (1988) Proceedings of the Fourth International Workshop on Campylobacter Infections (Kaijser, B. and Falsen, E., eds.) pp. 433-436, Gotema, Kungälv, Sweden.

Steininger, H., Faller, G., Dewald, E., Brabletz, T., Jung, A., and Kirchner, T. (1998) Virchows Arch. 433, 13-18.

Stellner, K., Saito, H. and Hakomori, S. -i. (1973) Arch. Biochem. Biophys., 155, 464-472.

Stellner, K. and Hakomori, S. -i. (1974) J. Biol. Chem., 249, 1022-1025.

Stroud, M. R., Handa, K., Salyan, M. E. K., Ito, K., Levery, S. B., Hakomori, S. -i., Reinhold, B. B. and Reinhold, V. N. (1996) Biochemistry, 35, 758-769.

Sung, J., Russell, R. I., Neyomans, Chan, F. K., Chen, S., Fock, K., Goh, K. L., Kullavanijaya,

P., Kimura, K., Lau, C., Louw, J., Sollano, J., Triadiafalopulos, G., Xiao, S., Brooks, P. (2000) J. Gastroenterol. Hepatol., 15, Suppl: G58-68.

Teneberg, S., Ångström, J., Jovall, P. -Å. and Karlsson, K. -A. (1994) J. Biol. Chem., 269, 8554-8563.

Teneberg, S., Lönnroth, I., Torres Lopez, J. F., Galili, U., Ölwegård Halvarsson, M., Ångström, J. and Karlsson, K. -A. (1996) Glycobiology, 6, 599-609.

Teneberg, S., Abul Milh, M., Lanne, B., Jovall, P. -Å., Karlsson, H., Ångström, J., Ölwegård Halvarsson, M., Danielsson, D., Ljung, Å., Wadström, T. and Karlsson, K. -A. (2001, manuscript in preparation).

Thorn, J. J., Levery, S. B., Salyan, M. E. K., Stroud, M. R., Cedergren, B., Nilsson, B., Hakomori, S. -i. and Clausen, H. (1992) Biochemistry, 31, 6509-6517.

Thurin, J., Brodin, T., Bechtel, B., Jovall, P. -Å., Karlsson, H., Strömberg, N., Teneberg, S., Sjögren, H. O. and Karlsson, K. -A. (1989) Biochim. Biophys. Acta, 1002, 267-272.

Vivier, E., Sorrell, J. M., Ackerly M., Robertson M. J., Rasmussen R. A., Levine H., and Anderson P. (1993) J. Exp. Med, 178(6), 2023-33.

Waldi, D. (1962) in Dünnschicht-Chromatographie (Stahl, E., ed.) pp. 496-515, Springer-Verlag, Berlin.

Watanabe, K. and Hakomori, S. -i. (1979) Biochemistry, 18, 5502-5504.

Wells, M. E. and Dittmer, J. C. (1963) Biochemistry, 2, 1259-1263.

Wotherspoon, A. C., Doglioni, C., Diss, T. C., Pan, L., Moschini, A., de Boni, M. and Isaacson, P. G. (1993) Lancet, 342, 575-577

Yamakawa, T. (1966) Colloq. Ges. Physiol. Chem., 16, 87-111.

Yang, H. -j. and Hakomori, S. -i. (1971) J. Biol. Chem., 246, 1192-1200.

What is claimed:

1. Use of a substance comprising Helicobacter pylori binding oligosaccharide sequence
[Gal(A)_{q}(NAc)ᵣ/Glc(A)_{q}(NAc)ᵣα3/β3]ₛ[Galβ4GlcNAcβ3]ₜGalβ4Glc(NAc)ᵤ
wherein q, r, s, t, and u are each independently 0 or 1,
so that when t=0 and u=0, then the oligosaccharide sequence is linked to a polyvalent carrier or present as a free oligosaccharide in high concentration, and analogs or derivatives of said oligosaccharide sequence having binding activity to Helicobacter pylori for the production of a composition having Helicobacter pylori binding or inhibiting activity.

2. The use according to claim 1, wherein said substance comprises the oligosaccharide sequence
GlcNAcβ3Galβ4GlcNAc or GlcNAcβ3Galβ4GlcNAcβ3Galβ4Glc
where position C4 of terminal GlcNAcβ3 is optionally linked to Galβ1- or an oligosaccharide chain by a glycosidic bond.

3. The use according to claim 1, wherein said substance comprises one or several of the following oligosaccharide sequences
Galβ4GlcNAc,
GalNAcα3Galβ4GlcNAc, GalNAcβ3Galβ4GlcNAc, GlcNAcα3Galβ4GlcNAc, GlcNAcβ3Galβ4GlcNAc, Galβ3Galβ4GlcNAc, Glcα3Galβ4GlcNAc, Glcβ3Galβ4GlcNAc,
Galβ4GlcNAcβ3Galβ4GlcNAc, Galβ4GlcNAcβ3Galβ4Glc,
GalNAcα3Galβ4GlcNAcβ3Galβ4Glc, GalNAcβ3Galβ4GlcNAcβ3Galβ4Glc, GlcNAcα3Galβ4GlcNAcβ3Galβ4Glc, GlcNAcβ3Galβ4GlcNAcβ3Galβ4Glc, Galβ3Galβ4GlcNAcβ3Galβ4Glc, Glcα3Galβ4GlcNAcβ3Galβ4Glc, Glcβ3Galβ4GlcNAcβ3Galβ4Glc,
GalANAcβ3Galβ4GlcNAc, GalANAcα3Galβ4GlcNAc, GalAβ3Galβ4GlcNAc, GalAα3Galβ4GlcNAc, GalANAcβ3Galβ4Glc, GalANAcα3Galβ4Glc, GalAβ3Galβ4Glc, GalAα3Galβ4Glc,
GlcANAcβ3Galβ4GlcNAc, GlcANAcα3Galβ4GlcNAc, GlcAβ3Galβ4GlcNAc, GlcAα3Galβ4GlcNAc, GlcANAcβ3Galβ4Glc, GlcANAcα3Galβ4Glc, GlcAβ3Galβ4Glc, GlcAα3Galβ4Glc,
Galβ4GlcNAcβ3Galβ4GlcNAcβ3Galβ4Glc, and reducing-end polyvalent conjugates thereof.

4. The use according to claim 1, wherein said substance comprises one or several of the following oligosaccharide sequences
GalNAcα3Galβ4Glc, GalNAcβ3Galβ4Glc, GlcNAcα3Galβ4Glc, GlcNAcβ3Galβ4Glc, Galβ3Galβ4Glc, Glcα3Galβ4Glc, Glcβ3Galβ4Glc, and reducing-end polyvalent conjugates thereof.

5. The use according to claim 3, wherein said substance comprises one or several of the following oligosaccharide sequences
Galβ4GlcNAcβ3Galβ4Glc (lacto-N-neotetraose),
Galβ4GlcNAcβ3Galβ4GlcNAcβ3Galβ4Glc (para-lacto-N-neohexaose), and reducing-end polyvalent conjugates thereof.

6. The use according to any one of claims 1-5, wherein said substance is conjugated to a polysaccharide, preferably to a polylactosamine chain or a conjugate thereof.

7. The use according to any one of claims 1-5, wherein said substance is a glycolipid.

8. The use according to any one of claims 1-5, wherein said substance is an oligomeric molecule containing at least two or three oligosaccharide chains.

9. The use according to any one of claims 1-5, wherein said substance consists of a micelle comprising one or more of the substances as defined in claims 1-8.

10. The use according to any one of claims 1-9, wherein said substance(s) is/are conjugated to a carrier.

11. The use according to any one of claims 1-10, wherein said substance is covalently conjugated with an antibiotic effective against Helicobacter pylori, preferably a penicillin type antibiotic.

12. The use according to claim 10, wherein position C1 of reducing end terminal Glc or GlcNAc of said oligosaccharide sequence (OS) is oxygen linked (—O—) to an oligovalent or a polyvalent carrier (Z), via a spacer group (Y) and optionally via a monosaccharide or oligosaccharide residue (X), forming the following structure
[OS—O—(X)ₙ—Y]ₘ-Z
where integers m, and n have values m≧1, and n is independently 0 or 1; X is preferably lactosyl-, galactosyl-, poly-N-acetyl-lactosaminyl, or part of an O-glycan or an N-glycan oligosaccharide sequence, Y is a spacer group or a terminal conjugate such as a ceramide lipid moiety or a linkage to Z;
or a derivative of the substance of said structure having binding activity to Helicobacter pylori.

13. Use of the substance as defined in claims 1-12 for the production of a pharmaceutical composition for the treatment or prophylaxis of any condition due to the presence of Helicobacter pylori.

14. The use according claim 13, wherein said pharmaceutical composition is for the treatment of chronic superficial gastritis, gastric ulcer, duodenal ulcer, gastric adenocarcinoma, non-Hodgkin lymphoma in human stomach, liver disease, pancreatic disease, skin disease, heart disease, or autoimmune diseases including autoimmune gastritis and pernicious anaemia and non-steroid anti-inflammatory drug (NSAID) related gastric disease, or for prevention of sudden infant death syndrome.

15. Use of the substance as defined in claims 1-12, for the diagnosis of a condition due to infection by Helicobacter pylori.

16. Use of the substance as defined in claims 1-12 for the production of a nutritional additive or composition for the treatment or prophylaxis of any condition due to the presence of Helicobacter pylori.

17. The use according to claim 16 wherein said nutritional additive or composition is for infant food.

18. Use of the substance as defined in claims 1-12, for the identification of bacterial adhesin.

19. Use of the substance as defined in claims 1-12 or a substance identified according to claim 18, for the production of a vaccine against Helicobacter pylori.

20. Use of the substance as defined in claims 1-12 for typing Helicobacter pylori.

21. Use of the substance as defined in claims 1-12 for Helicobacter pylori binding assays.

22. A Helicobacter pylori binding substance comprising an oligosaccharide sequence
Glc(A)_{q}(NAc)ᵣα3/β3 Galβ4Glc(NAc)ᵤ
wherein q, r and u are independently 0 or 1,
with the proviso that when said oligosaccharide sequence contains β3 linkage, both q and rare 0 or 1; or
GalA(NAc)ᵣα3/β3Galβ4Glc(NAc)ᵤ
wherein r and u are independently 0 or 1, and Helicobacter pylori binding analogs and derivatives thereof.

23. A Helicobacter pylori binding non-acidic polyvalent substance comprising the oligosaccharide sequence as defined in claim 1, wherein said oligosaccharide sequence (OS) is a part of structure
[OS—O—(X)ₙ—Y]ₘ-Z
as defined in claim 12, Y being a hydrophilic spacer, more preferably a flexible hydrophilic spacer, and Helicobacter pylori binding analogs and derivatives thereof.

24. The Helicobacter pylori binding non-acidic polyvalent substance according to claim 23, wherein linker structure Y is
[OS—O—(X)ₙ-L₁-CH(H/{CH₁₋₂OH}ₚ₁)—{CH₁OH}ₚ₂—{CH(NH—R)}ₚ₃—{CH₁OH}ₚ₄-L₂]ₘ-Z
wherein L₁ and L₂ are linking groups comprising independently oxygen, nitrogen, sulphur or carbon linkage atom or two linking atoms of the group forming linkages such as —O—, —S—, —CH₂-, —N—, —N(COCH3)-, amide groups CO—NH— or —NH—CO— or —N—N— (hydrazine derivative) or an amino oxy-linkages —O—N— and —N—O—; L1 is linkage from carbon 1 of the reducing end monosaccharide of X or when n=0, L1 replaces —O— and links directly from the reducing end C1 of OS; p1, p2, p3, and p4 are independently integers from 0-7, with the proviso that at least one of p1, p2, p3, and p4 is at least 1; CH₁₋₂OH in the branching term {CH₁₋₂OH}ₚ₁ means that the chain terminating group is CH₂OH and when the p1 is more than 1 there is secondary alcohol groups —CHOH— linking the terminating group to the rest of the spacer; R is preferably acetyl group (—COCH₃) or R is an alternative linkage to Z and then L₂ is one or two atom chain terminating group, in another embodiment R is an analog forming group comprising C₁₋₄ acyl group comprising amido structure or H or C₁₋₄ alkyl forming an amine; and m>1 and Z is polyvalent carrier; OS and X are as defined in claim 12.

25. A Helicobacter pylori binding substance comprising the oligosaccharide sequence
Gal(A)_{q}(NAc)ᵣ/Glc(A)_{q}(NAc)ᵣα3/β3Galβ4Glc(NAc)ᵤ
wherein q, r and u are each independently 0 or 1, with the proviso that said oligosaccharide sequence is not Galα3Galβ4Glc/GlcNAc,
as a non-reducing end terminal sequence, and Helicobacter pylori binding analogs and derivatives thereof.

26. The substance according to any one of claims 22-25 for use in binding bacteria, toxins or viruses.

27. The substance according to any one of claims 22-25 for use as a medicament.

28. A method for the treatment of a condition due to presence of Helicobacter pylori, wherein a pharmaceutically effective amount of the substance as defined in any one of claims 1-12 or 22-25 is administered to a subject in need of such treatment.

29. The method according to claim 28, when said condition is caused by the presence of Helicobacter pylori in the gastrointestinal tract of a patient.

30. The method according to claim 28, for the treatment of chronic superficial gastritis, gastric ulcer, duodenal ulcer, gastric adenocarcinoma, non-Hodgkin lymphoma in human stomach, liver disease, pancreatic disease, skin disease, heart disease, or autoimmune diseases including autoimmune gastritis and pernicious anaemia and non-steroid anti-inflammatory drug (NSAID) related gastric disease, or for prevention of sudden infant death syndrome.

31. The method of treatment according to any one of claims 28-30, wherein said substance is a nutritional additive or a part of a nutritional composition.

32. The substance according to claim 26, wherein said toxin is toxin a of Clostridium difficile.

33. The use according to claim 1, wherein said oligosaccharide sequence is β1-6 linked from the reducing end to GalNAc, GlcNAc, Gal or Glc.

34. The use according to claim 2, wherein said oligosaccharide sequence is
Glc(A)_{q}(NAc)ᵣβ3Galβ4GlcNAc
q and r being as defined in claim 1.
